(19) United States
(12) Patent Application Publication
(10) Pub. No.: US 2015/0231182 A1
(43) Pub. Date: Aug. 20, 2015

(54) CELL SYSTEMS AND METHODS FOR DELIVERING DISEASE-SPECIFIC THERAPIES

(71) Applicant: UNIVERSITY OF LOUISVILLE RESEARCH FOUNDATION, INC., Louisville, KY (US)

(72) Inventors: Nolan L. Boyd, Louisville, KY (US); Stuart K. Williams, Harrods Creek, KY (US); James B. Hoying, Louisville, KY (US); Venkat M. Ramakrishnan, Lexington, KY (US)

(21) Appl. No.: 14/426,618

(22) PCT Filed: Sep. 6, 2013
(86) PCT No.: PCT/US2013/058555

§ 371 Date: Mar. 6, 2015

Related U.S. Application Data
(60) Provisional application No. 61/698,306, filed on Sep. 7, 2012.

(51) Int. Cl. A61K 35/407; A61K 38/57; A61K 47/46; A61K 38/37

(52) U.S. Cl. CPC A61K 35/407; A61K 38/37; A61K 38/57; A61K 47/46; A61K 48/00

(57) ABSTRACT

Cell systems for delivering disease-specific therapies are provided that include a therapeutic cell combined with a plurality of stromal vascular fraction cells or a stromal vascular fraction cell-derived vasculature. The cell systems can include the therapeutic cells and the stromal vascular fraction cells in a biocompatible matrix or can further combine the therapeutic cells and stromal vascular fraction cells with microvessel fragments. Further provided are methods of treating a disease characterized by missing or defiicent gene products wherein a subject is administered an effective amount of a cell system that includes a therapeutic cell for supplying the missing or deficient gene products and a plurality of stromal vascular fraction cells.

### RELATED APPLICATIONS

This application claims priority from U.S. Provisional Application Ser. No. 61/698,306, filed Sep. 7, 2012, the entire disclosure of which is incorporated herein by this reference.

### TECHNICAL FIELD

The presently-disclosed subject matter relates to cell systems and methods of using the cell systems for delivering disease-specific therapies. In particular, the presently-disclosed subject matter relates to cell systems and methods that make use of therapeutic cells and stromal vascular fraction cells for providing disease-specific therapies to subjects.

### BACKGROUND

Inheritable diseases or genetic disorders that arise as a result of missing or deficient gene products affect millions of people worldwide and are often considered among the most difficult types of diseases to treat or protect against due to a lack of suitable curative or preventative therapies. For example, familial hypercholesterolemia (FH) has been observed to affect as many as 1 in 500 people, and commonly arises as a result of the FH subject either having one (heterozygous) or two (homozygous) non-functional low-density lipoprotein receptor (LDLR) genes. The lack of functional LDLR genes in these heterozygous and homozygous subjects results in elevated levels of cholesterol of 350-550 mg/dl to greater than 650 mg/dl, respectively, and subsequently causes the development of premature cardiovascular disease. In these subjects, cholesterol levels can, at least to a certain extent, be moderated by drug therapies (e.g. statins) and dietary control; however, some subjects, especially homozygous subjects, are refractory to treatment. For these subjects, only two treatment options are then available: (a) periodic apheresis treatments; or (b) a liver transplant.

With regard to these two different types of FH treatments, periodic apheresis treatments have been shown to be capable of significantly lowering cholesterol levels. Nevertheless, apheresis clinics are typically not widely available and, if the clinics are available, the apheresis treatments are expensive and require subjects to carve out 4 hours of more of time for treatments either weekly or biweekly, and then also manage the effects of cholesterol rebound. In this regard, the only “cure” that is currently available for FH has been liver transplantation, but, in addition to there being a shortage of available donor livers, liver transplantation procedures are typically not available to pediatric patients and also require life-long immune suppression. Recently, two other potential treatments have attempted to be developed as an alternative to liver transplantation, namely, gene therapy in the liver and delivery of therapeutic cells to the liver. Yet, to date, neither of these approaches have been effective for long term clinical resolution of FH.

One further potential treatment that has attempted to be developed as an alternative to liver transplantation in FH patients is tissue replacement. Indeed, tissue replacement is a potential strategy for regeneration of different tissues that are affected in a number of conditions involving organ failure and/or congenital abnormalities. However, minimal engraftment is often an issue with these approaches. Moreover, one of the major caveats in tissue replacement therapies is to promote effective vascularization of the transplanted tissue in order to prevent death and promote engraftment of transplanted cells. Several approaches have been utilized in this regard in an attempt to promote vascularization of implanted tissues, such as the delivery of angiogenic growth factors to recruit host vessels or co-implantation of endothelial and angiogenic signaling cells with target tissue cells. Nevertheless, and although considerable progress has been achieved to date, significant obstacles, such as the short half-life of growth factors in the tissues that results in the regression of newly formed vasculatures and the potential source of endothelial and angiogenic signaling cells for human transplants, still need to be addressed.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter relates to cell systems and methods of using the cell systems for delivering disease-specific therapies. In particular, the presently-disclosed subject matter relates to cell systems that include therapeutic cells and stromal vascular fraction cells and that are capable of forming a functional vasculature and inosculating with the vasculature of a subject to thereby provide a disease-specific therapy.

In some embodiments of the presently-disclosed subject matter, a cell system is provided for delivering disease-specific therapies that is comprised of a therapeutic cell and a plurality of stromal vascular fraction cells. In some embodiments, the cell system further comprises a microvessel fragment, such as one that is isolated from adipose tissue. In some embodiments, the therapeutic cells and the stromal vascular fraction cells are incorporated into a biocompatible matrix, such as, in some embodiments, a biocompatible matrix comprised of collagen. In some embodiments, the stromal vascular fraction cells are present in or are incorporated into the biocompatible matrix at a concentration of about 0.5×10⁶ to about 3.0×10⁶ cells/ml.

With regard to the therapeutic cells included in the cell systems of the presently-disclosed subject matter, in some embodiments, the therapeutic cells are isolated or wild-type parenchymal cells, such as, in some embodiments, a hepatocyte, a cardiomyocyte, or a pancreatic β-cell. In other embodiments, the therapeutic cell included in an exemplary cell system is an engineered therapeutic cell. In some embodiments, the engineered therapeutic cell includes one or more genetic modifications for providing missing or deficient gene products. For example, in certain embodiments, the engineered therapeutic cell is genetically-modified to express a low-density lipo-protein receptor (LDLR), such that the cell system can be implanted in a subject and used to treat elevated cholesterol levels in a subject. As another example, in some embodiments, the engineered therapeutic cell is genetically-modified to express clotting factor VIII, such that the cell system can be implanted in a subject and used to treat hemophilia A. As yet another example, in further embodiments, the engineered therapeutic cell is genetically-modified to express α1-antitrypsin, such that that cell system can be implanted in a subject and used to treat α1-antitrypsin deficiency in the subject.

In some embodiments of the cell systems, the engineered therapeutic cells included in the systems are derived from a stem cell. In some embodiments, the stem cell is an induced pluripotent stem cell that has been obtained by reprogramming a cell obtained from a subject. In such embodiments, the induced pluripotent stem cell can then be genetically-modified and differentiated into a desired therapeutic cell.

In some embodiments of the presently-disclosed subject matter, another exemplary cell system is provided in which the stromal vascular fraction portion of the cell system is not provided as individual cells, but is instead provided as a functional vascular assembly that is more readily capable of inosculation with a vasculature of a subject. In this regard, in some embodiments, a cell system for delivering disease-specific therapies is provided that comprises a therapeutic cell and a stromal vascular fraction cell-derived vasculature. In some embodiments, the therapeutic cell and the stromal vascular fraction cell-derived vasculature are incorporated into a biocompatible matrix.

Further provided by the presently-disclosed subject matter are methods of treating a disease characterized by a missing or deficient gene product. In some embodiments, a method of treating a disease characterized by missing or deficient gene products is provided that comprises administering to a subject in need thereof an effective amount of a cell system comprising a therapeutic cell for supplying the missing or deficient gene product and a plurality of stromal vascular fraction cells. In some implementations, the therapeutic cell and the plurality of stromal vascular fraction cells utilized in the therapeutic methods are incorporated into a biocompatible matrix. In some embodiments, administering the cell system comprises subcutaneously implanting or otherwise administering the cell system in a subject. In some embodiments, to increase the therapeutic effects of the cell systems, the administration of the cell system comprises subcutaneously administering or otherwise implanting the cell system at multiple sites in the body of a subject.

Still further provided, in some embodiments of the presently-disclosed subject matter, are kits. In some embodiments, a kit is provided that comprises a therapeutic cell and a plurality of stromal vascular fraction cells. In some embodiments, the kit comprises a first vessel including the therapeutic cells and a second vessel including the stromal vascular fraction cells. In some embodiments, the therapeutic cell and the plurality of stromal vascular fraction cells are incorporated into a matrix in the kit.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 is an image showing green fluorescent protein (GFP), rat adipose-derived stromal vascular fraction (SVF) cells formed into a functional microvasculature in vivo;

FIG. 2 is an image showing SVF vascularization in a disease model, where a SVF from C57BL/6-GFP mice was used to form a vasculature in a syngenic low-density lipoprotein receptor knockout (LDLR-KO) mouse;

FIG. 3 is a confocal microscopy image of a three-dimensional construct of rat SVF-GFP labeled with GS1 Biotin (for rodent endothelial cells) and counter-labeled with Streptavid-Cy5, where the construct was subsequently assessed for LDL-Dil uptake and HepG2-vascular interaction and co-localization was observed;

FIGS. 4A-4B include an image showing a vascularized insulin delivery organoid including pancreatic islets and microvascular fragments (FIG. 4A) and images showing the immunodetection of insulin in the organoid (Insuling-AF488), rodent endothelium (GS1-Rhodamine), and a merged image (merge) (FIG. 4B);

FIGS. 5A-5B include: images (FIG. 5A) showing adipose stromal vascular fraction cells formed into a perfused microvasculature in vivo, where fresh (fSVF) and cultured (cSVF) SVF isolated from GFP rats were seeded in 3-dimensional collagen type I gels and implanted subcutaneously into immunocompromised mice and where, after 4 weeks, host mice were perfused with dextran-TRITC through jugular injection; and graphs (FIG. 5B) showing vessel density (number of vessels/field of view), percentage of vessels perfused (*p=0.001), and average vessel diameter (*p=0.02);

FIGS. 6A-6B includes: images (FIG. 6A) showing angiogenesis with adipose stromal vascular fraction cells and showing that freshly isolated and cultured SVFs differ in their ability to incorporate into sites of neovascularization, where fSVF and cSVF isolated from GFP rats were co-implanted with microvessel fragments derived from non-GFP rats into immunocompromised mice for 14 or 28 days and were then removed and the vessels were stained with GS1-TRITC, where the black arrow shows SVF in endothelial cell position, and where the white arrows show SVF incorporated in perivascular position; and a graph (FIG. 6B) showing the quantification of SVF incorporation into neovessels 28 days post-implantation (percentage of total vessel volume);

FIG. 7 is a graph showing the expression of cell surface markers in freshly isolated (black bars) and cultured (white bars) SVF cells, where the cells were stained for the different molecules and analyzed by fluorescent flow cytometry, and where the percentage of cells positive for a specific molecule above isotype control is shown;

FIGS. 8A-8F are images showing that freshly isolated human adipose SVF cells vascularize implanted parenchymal cells, including images showing freshly isolated human SVF cells seeded in collagen type I gels, implanted subcutaneously into immunocompromised mice, and stained with UEA-TRITC after four weeks (FIGS. 8A-8C), and images showing human SVF and HepG2 bead constructs implanted for 6 weeks into the mice (FIGS. 8D-8F);

FIGS. 9A-9H are images and graphs showing that freshly isolated adipose SVF cells form a functional interface with implanted parenchymal cells that allows for Dil-LDL uptake, including: an image showing HepG2-GFP⁺ coated Cytodex-3 microcarrier beads (FIG. 9A); an image showing Dil-LDL within the construct (FIG. 9B); an image showing GS1-Cy5⁺ staining of murine endothelium and demonstrating formation of a vascular bed around beads (FIG. 9C); an image showing HepG2-GFP⁺ and Dil-LDL overlay showing co-localization (FIG. 9D); an image showing that HepG2-GFP⁺ coated Cytodex-3 microcarrier beads implanted without SVF cells do not form a GS1-Cy5⁺ vascular network as no Dil-LDL uptake was observed (FIG. 9E); and an image showing Dil-LDL uptake within host liver confirming adequate DiI-LDL delivery to host circulation (FIG. 9F); a graph showing the percentage overlap of HepG2-GFP⁺ clusters and GS1-Cy5⁺ vasculature and DiI-LDL in implants containing SVFs and HepG2-GFP⁺, where no HepG2 clusters lacking associated GS1-Cy5⁺ and DiI-LDL signal were identified (+) (FIG. 9G); and a scatter plot of implants with HepG2 clusters comparing Dil-LDL with GS1-Cy5⁺ vasculature (FIG. 9H);

FIG. 10 is a schematic diagram showing a pLenti vector with a LDLR insert, a CMV promoter, and sequence for Emerald GFP (EmGFP) co-expression;

FIGS. 11A-11B include images of undifferentiated induced pluripotent stem cell (iPSC)-derived hepatocyte-like cells (FIG. 11A, left) and induced pluripotent stem cell (iPSC)-derived hepatocyte-like cells after Stage 5 of differentiation (FIG. 11A, right), and images of a gel showing polymerase chain reaction (PCR) detection of albumin (ALB) transcription in iPSC, HLC at Stage 5, and HepG2, where beta-actin (ACTB) was used as a loading control.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method. In this regard, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Adipose-derived stromal vascular fraction (SVF) cells are a cell population that is obtained from the complete enzymatic digestion of adipose tissue to single cells followed by the discarding of the adipocytes. The SVF cells that result from the enzymatic digestion and discarding of adipocytes is thus a mix of heterogeneous cell populations that are composed of endothelial cells, fibroblasts, perivascular cells, immune cells, and undefined stem cell sub-populations. (see, e.g., Prockop, Science, 276:71-74, 1997; Theise et al., Hepatology, 31:235-40, 2000; Current Protocols in Cell Biology, Bonifacino et al., eds., John Wiley & Sons, 2000; and U.S. Pat. No. 4,963,489, which each describe stromal cells, including stromal vascular fraction cells and methods for isolating them, and which are each incorporated herein by this reference in their entirety). Despite the heterogenous nature of SVF cells, SVF cells have been identified for transplantation studies since adipose tissue (e.g., human adipose) is an easily accessible and dispensable tissue source that can provide large numbers of cells suitable for implantation with little donor morbidity and discomfort. In addition, it is appreciated that SVF cell preparations can be safely and effectively transplanted to either an autologous or allogeneic host and can be manufactured in accordance with Good Manufacturing/Tissue Practice guidelines. Indeed, the potential of SVF cells to promote vascularization and improve organ function when delivered to sites of ischemia has been demonstrated in animal models of peripheral ischemic disease and myocardial infarction. To date, however, the use of SVF cells as an effective means to provide disease-specific therapies has remained a major challenge.

To that end, the presently-disclosed subject matter is based, at least in part, on the discovery that adipose-derived SVF cells and adipose SVF cell-derived vasculatures can effectively integrate with the existing vasculature of a subject, interface with one or more therapeutic cells, and thereby provide disease-specific therapies. In particular, it has been determined that by making use of SVF cells and SVF cell-derived vasculatures, cell systems can be made for delivering disease-specific therapies that are non-immunogenic, modular, and retrievable as a platform for treating multiple diseases or disorders (e.g., genetic disorders). Further, these cell systems can provide for the integration of a therapeutic cell and, in some embodiments, an autologous therapeutic cell in a modular implantable format that, as described in detail below, is easily regulatable and removable. In addition to the inclusion of the therapeutic cells, the stromal vascular fraction components of these cell systems also allow for the generation of a vasculature for metabolic support and communication with the host. As such, rather than delivering a therapeutic gene or cell directly to a vital organ, such as the liver, the cell systems of the presently-disclosed subject matter can be implanted at easily accessible sites in the body of a subject by subcutaneous implantation and can then anastomose with the host vasculature to allow sufficient perfusion of the cell system and, consequently, the delivery of therapeutic molecules (including missing or defective gene products) into the blood stream of a subject or the lowering of toxins or other molecules to clinically relevant levels.

The presently-disclosed subject matter thus relates to cell systems and methods of using the cell systems for delivering disease-specific therapies. In particular, the presently-disclosed subject matter relates to cell systems that include therapeutic cells and stromal vascular fraction cells, and that are capable of forming a functional vasculature and inosculating with the vasculature of a subject to thereby deliver a disease-specific therapy. In some embodiments, a cell system for delivering disease-specific therapies is provided that includes a therapeutic cell and a plurality of stromal vascular fraction cells.

The term “therapeutic cell” is used herein to describe a cell that, when included in a cell system of the presently-disclosed subject matter, is capable of providing for the “treatment” of a specific disease or disorder as defined herein below. In some embodiments, the therapeutic cell is an isolated parenchymal cell that typically comprises the functional part of a particular tissue or organ, but that may be missing or dysfunctional in a subject afflicted with a particular disease or disorder. In this regard, exemplary types of parenchymal cells that can be incorporated into the cell systems of the presently-disclosed subject matter to provide a therapeutic effect include neurons, cardiomyocytes, myocytes, chondrocytes, pancreatic acinar cells, islets of Langerhans (including pancreatic β cells), osteocytes, hepatocytes, Kupffer cells, fibroblasts, myoblasts, satellite cells, endothelial cells, adipocytes, preadipocytes, biliary epithelial cells, and the like. Each of these types of cells can be isolated and cultured by conventional techniques known in the art and then included in a cell system in accordance with the presently-disclosed subject matter. Such exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022. In some embodiments, the parenchymal cell that is incorporated into an exemplary cell system is a hepatocyte, a cardiomyocyte, or a pancreatic β cell.

In some embodiments of the presently-disclosed cell systems, the therapeutic cell is a not an isolated wild-type cell or parenchymal cell that is typically found in a particular tissue or organ, but is instead an engineered therapeutic cell. The term “engineered therapeutic cell” is used herein to describe cells that are modified either structurally or functionally to provide for the “treatment” of a specific disease as described herein below. For example, in some embodiments, the engineered therapeutic cell includes one or more genetic modifications for providing gene products that are missing or deficient in a particular disease or disorder.

The term “gene” is used broadly herein to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters affecting the expression or function of the gene. As such, the term “gene product” is used herein to refer to any biochemical material, such as RNA or protein, resulting from the expression of a gene.

As used herein, the term “genetic modification” is used to refer to any manipulation of an organism's genetic material in a way that does not occur under natural conditions. Methods of performing such manipulations are known to those of ordinary skill in the art and include, but are not limited to, techniques that make use of vectors for transforming cells with a nucleic acid sequence of interest. In this regard, the term “vector” is used herein to refer to any vehicle that is capable of transferring a nucleic acid sequence into a cell. For example, vectors which can be used in accordance with the presently-disclosed subject matter include, but are not limited to, plasmids, cosmids, bacteriophages, or viruses, which can be transformed by the introduction of a nucleic acid sequence of interest for use in the cells systems of the presently-disclosed subject matter. Such vectors are well known to those of ordinary skill in the art.

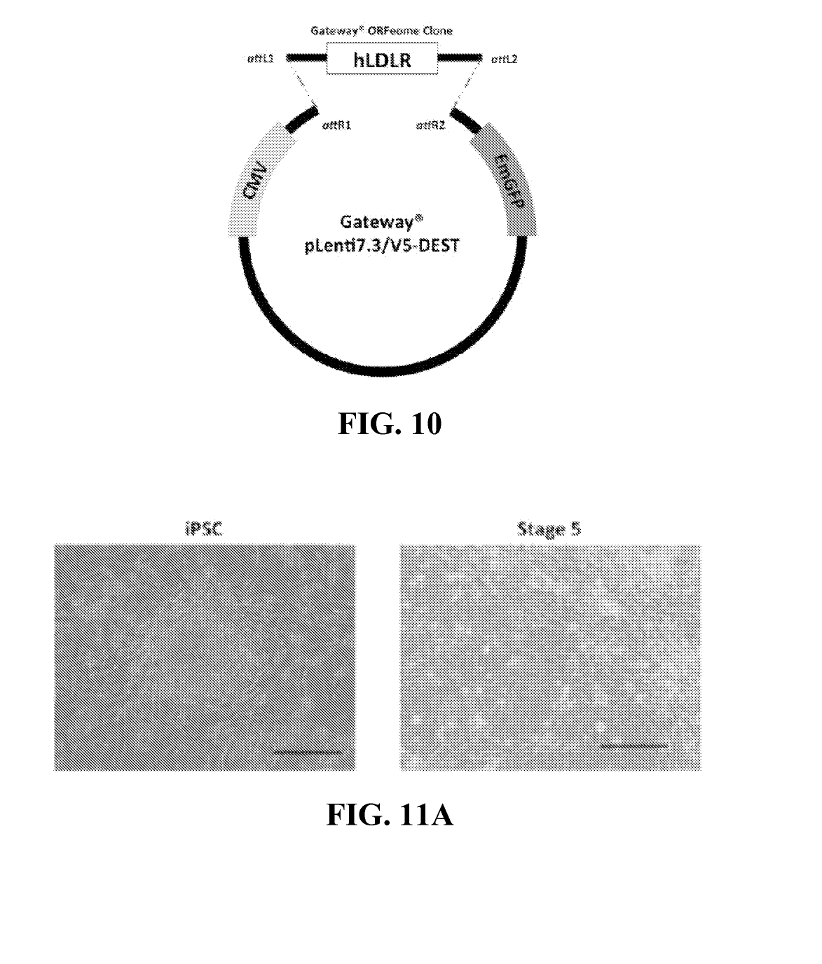

As one exemplary embodiment of a vector comprising a nucleic acid sequence of the presently-disclosed subject matter, an exemplary vector can be a plasmid or viral construct into which a nucleic acid encoding a low-density lipoprotein receptor (LDLR) polypeptide can be cloned by the use of internal restriction sites present within the vector. For example, in some embodiments, a episomal plasmid (pEHZ-LDLR-LDLR) can be used that contains 10 kb of upstream regulatory sequences for physiological control of LDLR expression (see Hibbitt, et al., Long-term Physiologically Regulated Expression of the Low-density Lipoprotein Receptor In Vivo Using Genomic DNA Mini-gene Constructs, Molecular Therapy (2010) 18(2), 317-326, which is incorporated herein by this reference). In other embodiments, a lentivirus construct may be utilized containing the human LDLR (see, e.g., FIG. 10).

Regardless of the particular vector utilized, the nucleic acids that are inserted into an exemplary engineered therapeutic cell of the presently-disclosed subject matter are typically operably linked to an expression cassette. The terms “associated with,” “operably linked,” and “operatively linked” refer to two nucleic acid sequences that are related physically or functionally. For example, a promoter or regulatory nucleic acid sequence is said to be “associated with” a nucleic acid sequence that encodes an RNA or a polypeptide of interest if the two sequences are operatively linked, or situated such that the regulator nucleic acid sequence will affect the expression level of the coding or structural nucleic acid sequence.

The term “expression cassette” refers to a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression.

In some embodiments, to control the amount of expression of a nucleic acid sequence of interest (e.g., a nucleic acid sequence encoding a gene that is missing or defective in a particular disease state) an expression cassette is provided that further comprises a promoter for expressing the nucleic acid sequence of interest at a desired level. As would be recognized by those skilled in the art, a “promoter” is a control sequence that is a region of a nucleic acid sequence at which initiation and rate of transcription are controlled. The phrases “operatively positioned,” “operatively linked,” “under control,” and “under transcriptional control,” when used in reference to a promoter, mean that a promoter is in a correct functional location and/or orientation in relation to a nucleic acid sequence to control transcriptional initiation and/or expression of that sequence. A promoter also may or may not be used in conjunction with an “enhancer,” which refers to a cis-acting regulatory sequence involved in the transcriptional activation of a nucleic acid sequence.

As noted, in some embodiments of the presently-disclosed subject matter, the engineered therapeutic cells contain one or more genetic modifications that are designed to increase the expression of a protein or other gene product known to be missing or deficient in a particular disease or disorder of interest. As one example of such an engineered therapeutic cell, in some embodiments, an engineered therapeutic cell is provided that is genetically modified to express a low-density lipo-protein receptor (LDLR), such that the engineered cell product can be used in cell system that is administered to subjects suffering from familial hypercholesterolemia and who exhibit a decreased expression of the LDLR. In this regard, and as described in more detail below, such a cell system thus provides the LDLR in a modular format that can be administered to a subject to continuously clear cholesterol and can be adjusted by varying the number of therapeutic cells or number of cell systems administered to a subject in order to meet clearance requirements for that particular subject. Moreover, such a cell system also avoids complications of direct virus administration or hepatocyte delivery to the liver, while also facilitating clinical observation and rapid removal that is not possible with direct therapeutic treatments to the liver. Additionally, although the target of such a cell system is cholesterol removal in subjects with defective or missing LDLRs, because of the direct correlation of high cholesterol levels with cardiovascular disease (CVD) development, the clinical benefit of the such cell systems is also a reduction in cholesterol-related downstream diseases that would also be useful for non-FH subjects as well.

As another example of a cell system including engineered therapeutic cells designed to increase the expression of a missing or deficient protein or other gene product, in some embodiments, an engineered therapeutic cell is provided that is genetically modified to express clotting factor VIII, such that the engineered cell product can be used in subjects suffering from hemophilia A. As yet another example, in some embodiments, the engineered therapeutic cell is genetically modified to express al-antiptrypsin, such that the engineered therapeutic cell can be used in subjects suffering from a disease or disorder arising as a result of an α1-antiptrypsin deficiency.

With further regard to the engineered therapeutic cells used in the cell systems of the presently-disclosed subject matter, in some embodiments, the missing or defective gene products expressed by the engineered therapeutic cells are naturally or artificially configured such that the gene products are retained by the engineered therapeutic cells or are released from the cells. For instance, and with reference to the foregoing examples of engineered therapeutic cells, LDLR proteins are naturally retained by the therapeutic cells and used to take cholesterol-rich LDL molecules into the cells, while clotting factor VIII and α1-antiptrypsin are typically naturally released from the cells to exert their therapeutic effects. Of course, if it is desired to modify a particular gene product such that it is released by a cell when it is typically retained or such that it is retained by a cell when it is typically released, such methods for modifying nucleic acid sequences to cause the expressed gene products to be artificially retained by or released from a particular cell are well known to those skilled in the art and can be applied to a particular nucleic acid sequence in accordance with the presently-disclosed subject matter as desired.

Any type of cell that is capable of being transformed with and expressing a nucleic acid sequence of interest and that is then capable of being combined with a stromal vascular fraction can be used. In this regard, in some embodiments, the engineered therapeutic cells can be comprised any cell that can be derived from pluripotent cells (i.e., embryonic or induced). Such cells include, but are not limited to, hepatocytes, Ito cells, Kupffer cells, fibroblasts, mesenchymal stromal cells, endothelium, cholangiocytes, ependymal cells, astrocytes, Schwann cells, smooth muscle, neurons, cardiac fibroblasts, or cardiomyocytes. Further, in some embodiments, any of the foregoing types of cells, can be isolated from the tissue of a subject and used to produce an engineered therapeutic cell in accordance with the presently-disclosed subject matter.

In some embodiments of the presently-disclosed subject matter, the engineered therapeutic cell is derived from a stem cell, such as an induced pluripotent stem cell (iPSC), that can be transfected with a vector including a nucleic acid sequence of interest, and then differentiated into a mature, differentiated phenotype (i.e., the phenotype of a parenchymal cell found in a particular tissue) that is then capable of expressing a gene product encoded by the nucleic acid sequence. As used herein, the term “stem cells” refers broadly to traditional stem cells, progenitor cells, preprogenitor cells, precursor cells, reserve cells, and the like. Exemplary stem cells include, but are not limited to, embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387-403; Pittinger et al., Science, 284:143-47, 1999; Animal Cell Culture, Masters, ed., Oxford University Press, 2000; Jackson et al., PNAS 96(25):14482-86, 1999; Zuk et al., Tissue Engineering, 7:211-228, 2001; and U.S. Pat. Nos. 5,559,022, 5,672,346 and 5,827,735.

In some embodiments of the cell systems of the presently-disclosed subject matter, the engineered therapeutic cell that is included in an exemplary cell system is derived from a cell (e.g., an SVF cell) that has been reprogrammed into an iPSC cell, genetically modified to express a nucleic acid sequence of interest, and then subsequently differentiated into a desired parenchymal cell expressing the nucleic acid sequence of interest. For instance, in certain embodiments, SVF cells or fibroblast cells can be obtained from a subject and then reprogrammed into the iPSCs by transfecting the cells with vectors encoding the reprogramming genes POU5F1, NANOG, SOX2 and MYC. The resulting iPSCs can then transfected with a nucleic sequence of interest (e.g., a vector encoding a LDLR) and can subsequently be differentiated into a variety of different parenchymal cell types using methods known to those skilled in the art (see, e.g., Song, et al. “Efficient Generation of Hepatocyte-Like Cells from Human Induced Pluripotent Stem Cells,” Cell Research (2009) 19: 1233-1242, which describes the generation of hepatocyte-like cells from iPSCs and which is incorporated herein by this reference in its entirety).

With further regard to the components of an exemplary cell system of the presently-disclosed subject matter, in some embodiments and in addition to including stromal vascular fraction cells in an exemplary cell system, the cell systems further comprise a microvessel fragment to further facilitate the formation of a functional microvasculature and to facilitate the inosculation of the functional vasculature of the cell system with the vasculature of a host. The terms “vascular fragment” and “vessel fragment” are used interchangeably herein to refer to a segment or piece of vascular tissue, including at least a part or segment of at least an artery, arteriole, capillary, venule, vein, or a combination thereof. As such, the terms vascular fragment or vessel fragment are further inclusive of the terms “microvessel fragment” or “microvascular fragment,” which are used interchangeably herein to refer to a segment or piece of a smaller caliber vascular tissue, such as arterioles, capillaries, and venules. Typically, a vessel or microvessel includes endothelial cells arranged in a tube surrounded by one or more layers of mural cells, such as smooth muscle cells or pericytes, and can further comprise extracellular matrix components, such as basement membrane proteins. In some embodiments, the vascular fragments are obtained from vascular tissue, such as that found in skin, skeletal muscle, cardiac muscle, the atrial appendage of the heart, lung, mesentery, or adipose tissue. In some embodiments, the vascular fragments are adipose tissue microvessel fragments that can be isolated or otherwise obtained from the incomplete digestion of various adipose tissues including, but not limited to, subcutaneous fat, perirenal fat, pericardial fat, omental fat, breast fat, epididymal fat, properitoneal fat, and the like.

To combine the therapeutic cells, stromal vascular fraction cells, and, if present in a particular cell system, microvessel fragments into a modular construct that facilitates the formation of a functional vasculature and that can be easily placed or administered to a subject, in some embodiments, the components of the cell systems of the presently-disclosed subject matter are incorporated into or otherwise included into a biocompatible matrix. The term “biocompatible” is used herein to refer to a matrix that is substantially non-toxic in the in vivo environment of its intended use, and that is not substantially rejected by the subject's physiological system (i.e., is non-antigenic). As will be recognized by those of ordinary skill in the art, the biocompatibility of a particular matrix can be gauged by the matrix's toxicity, infectivity, pyrogenicity, irritation potential, reactivity, hemolytic activity, carcinogenicity, and/or immunogenicity. When introduced into a majority of subjects, a biocompatible matrix will not cause an undesirably adverse, long-lived, or escalating biological reaction or response, and is distinguished from a mild, transient inflammation, which typically accompanies surgery or implantation of foreign objects into a living organism.

In certain embodiments, the components of an exemplary cell system are placed in a biocompatible matrix by first isolating the stromal vascular fraction cells and, if present, microvessel fragments from adipose tissue and then combining the stromal vascular fraction cells, microvessel fragments, and desired therapeutic cells with a liquid, unpolymerized matrix material, such as cold, unpolymerized collagen, fibrin, or other nonpolymerized matrix materials, or the like. Once the cell system components and non-polymerized matrix material have been combined, the unpolymerized construct can then be placed into a suitable vessel, such and allowed to polymerize into a three-dimensional construct that can then be inserted into a subject to provide disease-specific therapies. In this regard, in some embodiments, the term “cell system” can be used interchangeably with the terms “tissue construct,” “construct,” “tissue mimic,” or “organoid.”

In some embodiments, the stromal vascular fraction cells can be placed in the biocompatible matrix such that the stromal vascular fraction cells are present in the matrix at a concentration of about 0.5×10⁶ cells/ml, about 1.0×10⁶ cells/ml, about 1.5×10⁶ cells/ml, about 2.0×10⁶ cells/ml, about 2.5×10⁶ cells/ml, or about 3.0×10⁶ cells/ml. In further embodiments, upon combining the cell system components in the biocompatible matrix, the cell system can then be cultured for a sufficient period of time such that the stromal vascular fraction cells and, if present, the microvessel fragments within the cell system can form or, at the least, begin to form a functional vasculature before being administered to a subject. In this regard, and as indicated above, a cell system can be provided in some embodiments that includes a therapeutic cell, a stromal vascular fraction cell-derived vasculature, and, optionally, a microvessel fragment-derived vasculature.

With further regard to the cell systems in which the components are incorporated into a biocompatible matrix, one of ordinary skill in the art will understand that such constructs, when provided in a non-polymerized form and subsequently allowed to polymerize or gel, are capable of assuming a multitude of sizes and shapes. Thus, in certain embodiments, the ultimate size and shape of the polymerized construct depends, in part, on the size and shape of the vessel in which the construct is polymerized. Of course, to the extent it may be desired, different sizes or shapes of constructs can be provided by altering the geometry of the centrally-disposed cavity of the exemplary biochamber, or other vessel, into which the unpolymerized construct is placed. Additionally, in certain embodiments, polymerized constructs can be cut or trimmed into a desired size or shape. Thus, constructs can be prepared in virtually any size and shape and can include any desired number of therapeutic cells or stromal vascular fraction cells, as may be appropriate for a particular application or therapy.

Further provided, in some embodiments of the presently-disclosed subject matter, are methods of treating diseases that are characterized by missing or deficient gene products. In some embodiments, a method of treating a disease characterized by missing or deficient gene products is provided that comprises administering to a subject in need thereof an effective amount of a cell system comprising a therapeutic cell for supplying the missing or deficient gene product and a plurality of stromal vascular fraction cells. In this regard, by providing a cell system having a therapeutic cell that provide the gene product that is missing in a particular disease state, the cell systems of the presently-disclosed subject matter are configured to provide a treatment that is specific for or matched to that particular disease or disorder.

As used herein, the terms “treatment” or “treating” relate to any treatment of a condition of interest (e.g., a diseases characterized by missing or deficient gene products), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

In some embodiments of the presently-disclosed methods, the cell systems are used to treat familial hypercholesterolemia by providing a cell system that includes an engineered therapeutic cell genetically-modified to express a low-density lipo-protein receptor (LDLR). In this regard, such a cell system can be administered to a subject, allowed to inosculate with the existing vasculature of the subject, and then utilized to mediate the endocytosis of cholesterol rich-LDL molecules from the blood stream of the subject. In other words, by administering such a cell system to a subject, the cell system can effectively be used as an apheresis system to lower the cholesterol levels in the blood stream of a subject with familial hypercholesterolemia.

Of course, the cell systems of the present-disclosed subject matter are not limited to diseases and disorders in which the scavenging of cholesterol is desired, but can also be used to treat any disease or disorder where a missing or defective gene product is the underlying cause of the disease or disorder. For example, in other embodiments, the cell systems are used to treat hemophilia A by providing a cell system including an engineered therapeutic cell genetically modified to express clotting factor VIII.

Suitable methods for administering a therapeutic cell system in accordance with the methods of the presently-disclosed subject matter include, but are not limited to parenteral administration (including intravascular, intramuscular, and/or intraarterial administration), subcutaneous administration, intraperitoneal administration, surgical implantation, and local injection. In some embodiments, the cell systems of the presently-disclosed subject matter are implanted in a subject, such as by, in some embodiments, subcutaneous administration. In some embodiments, subcutaneously administering the cell system comprises subcutaneously administering one or more cell systems at multiple sites in the body of a subject.

Regardless of the particular route of administration, the cell systems of the presently-disclosed subject matter are typically administered in amount effective to achieve the desired response. As such, the term “effective amount” is used herein to refer to an amount of the therapeutic cell system (e.g., a cell system comprising engineered therapeutic cells genetically modified to express LDLR and a plurality of stromal vascular fraction cells) sufficient to produce a measurable biological response (e.g., a decrease in the amount of a LDL or cholesterol). Actual amounts of therapeutic cells or the amount of expression of a particular gene product in an engineered therapeutic cell in a cell system of the presently-disclosed subject matter or the number of cell systems used for a particular treatment can be varied so as to administer an amount of the active therapeutic cells(s) that is effective to achieve the desired therapeutic response for a particular subject and/or application. Of course, the effective amount in any particular case will depend upon a variety of factors including the activity of the therapeutic cells, formulation, the route of administration, combination with other treatments, severity of the condition being treated, and the physical condition and prior medical history of the subject being treated. Preferably, a minimal amount is administered, and the amount is escalated in the absence of dose-limiting toxicity to a minimally effective amount. Determination and adjustment of a therapeutically effective amount, as well as evaluation of when and how to make such adjustments, are known to those of ordinary skill in the art.

Still further provided, in some embodiments of the presently-disclosed subject matter, are kits that comprise a cell system including a therapeutic cell and a plurality of stromal vascular faction cells. In some embodiments, the kit can be provided with a first vessel including the therapeutic cells and a second vessel including the stromal vascular fraction cells. In other embodiments of the kits, the therapeutic cells and the stromal vascular fraction cells are combined and incorporated into a biocompatible matrix, such that the kit provides an assembled cell system that can readily be administered to a subject. In some embodiments, the kit can further include one or more microvessel fragments or the materials for producing a biocompatible matrix. In some embodiments, a kit comprising a cell system of the presently-disclosed subject matter is provided along with instructions for combining the components to produce a cell system and/or with instructions for using the cell system in a subject.

With respect to the presently-disclosed subject matter, a preferred subject is a vertebrate subject. A preferred vertebrate is warm-blooded; a preferred warm-blooded vertebrate is a mammal A preferred mammal is most preferably a human. As used herein, the term “subject” includes both human and animal subjects. Thus, veterinary therapeutic uses are provided in accordance with the presently-disclosed subject matter. As such, the presently-disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N Y ; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. Some of the following examples are prophetic, notwithstanding the numerical values, results and/or data referred to and contained in the examples. Furthermore, the following examples may include compilations of data that are representative of data gathered at various times during the course of development and experimentation related to the present invention.]

### EXAMPLES

### Example 1—Use of Genetically Autologous Stromal Vascular Fraction Cells to Form a Functional Vasculature in a Disease Model

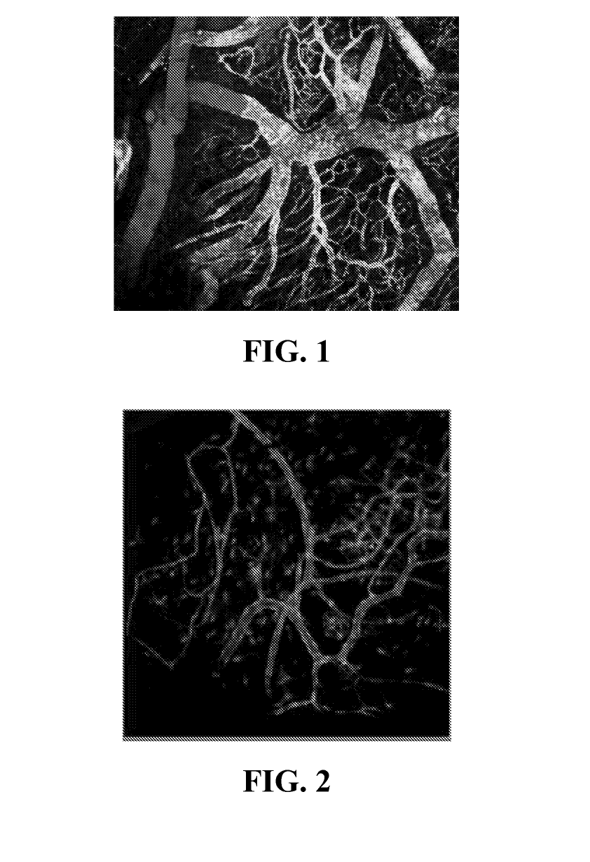

The development of an engineered organoid or cell system not only requires a therapeutic parenchymal cell, but also a functional vasculature that can interact with the host and not induce an immune response. Adipose tissue is highly vascularized and can be digested to separate the adipose cells from the remaining stroma, which is also referred to as the stromal vascular fraction (SVF). When SVF cells are added to a three-dimensional collagen I construct and implanted subcutaneously, the SVF cells self-assemble into a functionally mature vasculature (see, e.g., FIG. 1). As such, it was thought that SVF cells are a microvascular regenerative system and can be used as an autologous source for vascularizing organoids, and experiments were undertaken to test if SVF cells could be used to generate a vasculature in a genetic disease model, specifically the low-density lipoprotein receptor-knock out mouse (LDLR-KO). The LDLR-KO mouse does not express the LDLR and therefore does not clear cholesterol via the LDLR mechanism. In this regard, even when not fed a high fat diet, the LDLR-KO mouse has 2-3 times the normal level of circulating LDL.

Briefly, in these experiments, adipose tissue was first isolated from the epididymal fat pad or uterine horn fat of C57BL/6-green fluorescent protein (GFP) mice (the same genetic background as the LDLR-KO and therefore genetically autologous), digested, and centrifuged to separate the adipose and SVF cells. SVF-GFP cells were resuspended in collagen I (3 mg/ml) at a concentration of 10⁶/ml and implanted subcutaneously in the backs of LDLR-KO mice (2 constructs/mouse) for 6 weeks. The constructs were subsequently harvested and imaged using an Olympus confocal microscope.

Upon analysis of the results, it was observed that SVF cells from mice constitutively expressing GFP permit cell tracking in the implant construct. The genetically autologous SVF-GFP cells were able to self-assemble into a functional vasculature (FIG. 2) in spite of the elevated LDL. This indicated adipose SVF-cells could be isolated from a subject with a gene defect, the disorder corrected, and SVF cells used for generating a therapeutic vasculature. Moreover, the results indicated that adipose-derived SVF cells are capable of self-assembly into a functional vasculature, further indicating it is a microvascular regenerative system and can be useful for therapeutic vascularization applications in diseases involving genetic disorders.

### Example 2—Development of Engineered Implantable Vascularized Cell-Based LDL Apheresis System Using Hepatocytes

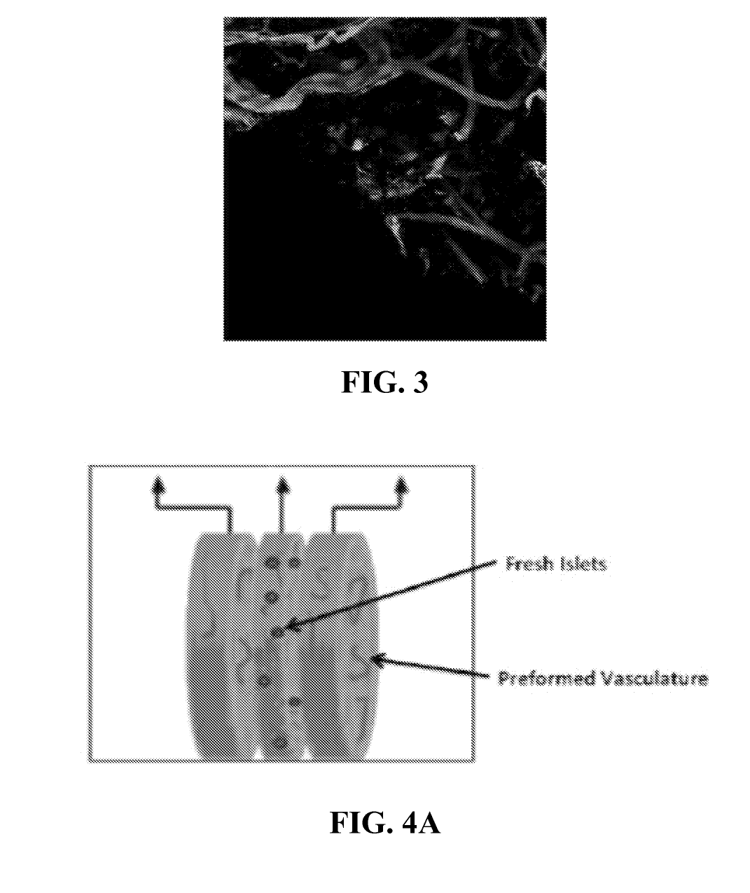

Familial hypercholesterolemia is characterized by pathologically elevated LDL-cholesterol due to LDL-receptor (LDLR) gene defects. In this regard, it was thought that an implantable cell-based apheresis system can scavenge excessive LDL cholesterol, and experiments were undertaken to design a strategy that combines adipose stromal vascular fraction cells (SVF) for stromal and vascular support with hepatocyte model cells (HepG2) for LDL clearance. To begin the development of such a strategy, LDLR induction in HepG2 was first assessed by serum starving the cells for 48 h, followed by exposure to 1, 0.2, 2, or 20 μM of Lovastatin. Maximal LDLR expression was observed with the 20 μM treatment. HepG2-coated Cytodex-3 beads were then placed within 3 mg/mL collagen constructs containing SVF cells, which was expected to sustain HepG2 cells and form robust host-construct vascular associates. Constructs were then bilaterally implanted in Rag-1 deficient mice (sacrificed at 2, 4, and 6 weeks). In vivo, HepG2 LFLR expression was then enhanced by bilaterally injecting Lovastatin subcutaneously 48 h and 24 h before sacrifice. LDL-Dil (50 μg) was injected via the tail vein 24 h prior to sacrifice. Explanted constructs, labeled with GS1 Biotin (for rodent endothelial cells) and counter-labeled with Streptavid-CyS, were subsequently assessed for LDL-Dil uptake and HepG2-vascular interaction by confocal microscopy and histology, and co-localization was observed (see, e.g., FIG. 3).

In the development of a cell-based implantable apheresis system, adipose-derived SVF cells were utilized for several reasons, namely: SVF cells were an autologous cell source that were functionally similar to BM-MSC; SVF cells were more readily accessible and in larger quantities than other potential autologous choices; SVF cells were functionally useful as either fresh isolate or after culture; adipose transfer for reconstruction has been approved for clinical use and devices are being developed for rapid isolation for clinical uses; and both fresh and cultured SVF cells can form a functional microvasculature in vivo. More specifically, when a SVF single-cell suspension (rat, mouse and human) is implanted in three-dimensional collagen I constructs, the SVF cells self-assemble into a new vasculature as shown in FIG. 1, where green fluorescent protein (GFP) transgenic rat SVF cells (10⁶/ml) were dispersed into a collagen I construct (3 mg/ml) and implanted subcutaneously in an immune-compromised mouse (B6.129S7-Rag1^{tm1Mom}/J) for 4 weeks. In other words, the SVF cells can act as an autologous organoid vascularization source.

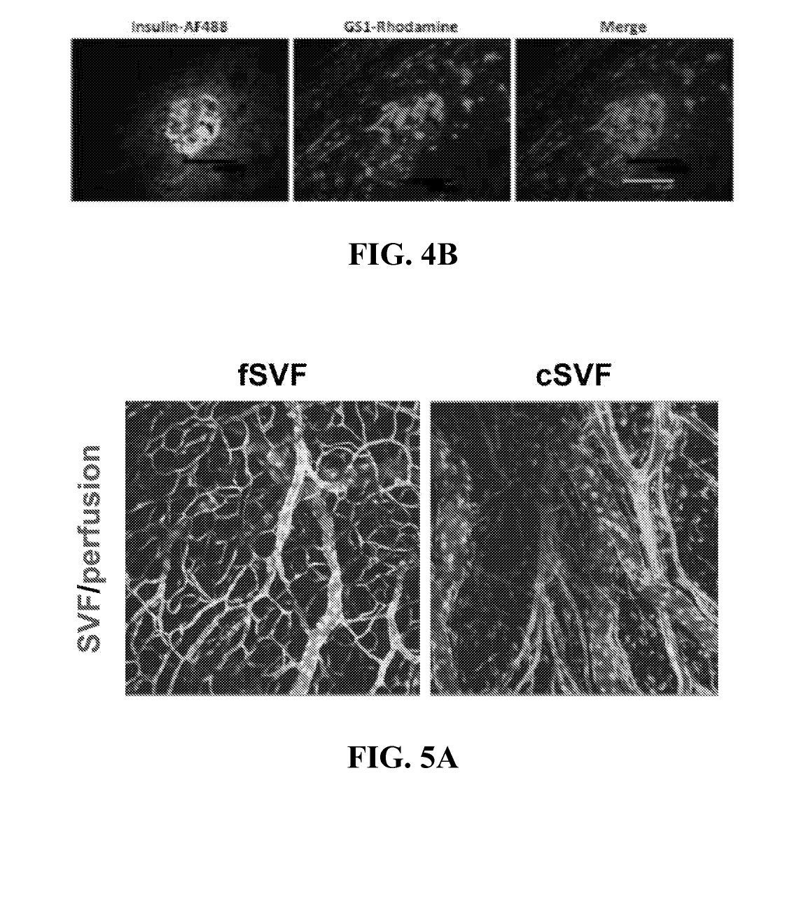

With further regard to the use of SVF cells in the development of cell systems and, in particular, organoids, a vascularized organoid was also been engineered and fabricated for delivery of insulin (FIG. 4A). Briefly, micro-vessel fragments (MVF, incomplete SVF digestion and isolation of residual vessel fragments) were initially isolated from rat-SVF and pre-cultured in 3D collagen I in vitro to form vascularized constructs. Islets were then freshly isolated, cast into 3D constructs, and subsequently sandwiched on both sides with the preformed MVF constructs (FIG. 4A). Although MVF and not SVF were used in that experiment, the islets were observed to survive implantation and produced insulin (FIG. 4B, first panel). The vascularized construct also rapidly anastomosed with the host (FIG. 4B, middle panel) and perfused the construct, which supported the islet's metabolic needs (FIG. 4B, merge).

### Example 3—Generation of a Functional Liver Tissue Mimic Using Adipose Stromal Vascular Fraction Cell-Derived Vasculatures

To harness the vascularization potential of SVF cells in vivo and to generate an effective vascular interface between host and transplanted liver cells that resulted in a functional tissue mimic, experiments were undertaken to determine: (1) whether adipose-derived SVF cells have a potent intrinsic vascularizing potential; (2) whether culturing freshly isolated SVF cells retained that vascularization potential despite possible changes in cell populations; and (3) whether SVF cell-derived vasculatures formed a functional interface between host and implanted parenchymal cells.

Materials and Methods

For SVF isolation, adipose-derived SVF cells were isolated from the epididymal fat pads of male, retired breeder Sprague-Dawley rats (Charles River) under anesthesia [ketamine (40-80 mg/kg) and xylazine (5-10 mg/kg)]. Green fluorescent protein (GFP)-tagged SVF were obtained from Sprague-Dawley rats that ubiquitously express GFP (Rat Research and Resource Center, University of Missouri, Columbia, Mo.). Human SVF were isolated from adipose tissue obtained from abdominoplasty. Harvested fat was washed in 0.1% BSA-PBS, finely minced, and digested in 2 mg/ml type I collagenase solution (Worthington Biochemical Company, Freehold, N.J., USA) for 40 min at 37° C. with vigorous shaking. Adipocytes were removed by centrifugation, and the entire cell pellet was washed with 0.1% BSA-PBS. Cells were either immediately used (Fresh SVF, fSVF) or plated into gelatin-coated plates (Cultured SVF, cSVF 5×10⁴ cells/cm²) in fresh media (DMEM supplemented with 2 mM L-glutamine, 50 μg/ml ECGS and 10% FBS). Cultured SVF were used at P0 after 5-7 days when cells reached confluence.

For flow cytometry analysis, cSVF were first dissociated with non-enzymatic Cell Dissociation Buffer (Sigma) after reaching confluence (P0) and fixed with 4% paraformaldehyde for 10 min at room temperature. Cells were blocked with PBS containing 5% fetal bovine serum (FBS) for 30 minutes on ice and incubated with the following antibodies in blocking buffer on ice for 1 hour: anti-CD14 (1:100), anti-CD31-APC (1:500, BD Biosciences); anti-cKit (1:100, Abcam); anti-CXCR4 (1:100, Ebiosciences); anti-c-Met (1:100), anti-PDGFR-β (1:100, Santa Cruz Biotechnology) overnight at 4° C. Secondary antibodies used were anti-mouse-Alexa Fluor 488 (1:400, Jackson ImmunoResearch) and anti-rabbit-Cy5 (1:500, Jackson ImmunoResearch) for 30 min at 4° C.

For microvessel isolation, fat-derived microvessels (FMF) were isolated from rat epididymal fat by limited collagenase digestion and selective screening as previously described. The collagenase used (type I; Worthington Biochemical Company, Freehold, N.J., USA) was lot tested to yield high numbers of fragments with intact morphologies. These vessel fragments have the potential to form a microcirculation composed of different vessel types 4 weeks post implantation in vivo in 3-dimensional collagen gels.

HepG2 cells were also cultured in T-75 tissue culture flasks in HepG2 growth media consisting of Dulbecco's Modified Eagle's Media high glucose, 10% fetal bovine serum, 1× penicillin/streptomycin, and 1× L-glutamine (Invitrogen Camarillo, Calif., USA). Media was changed every other day and cells were grown to confluence at which time they were prepared for Cytodex-3 culture as described below. Plasmids and Cell Transduction HepG2 were transduced with retrovirus to constitutively express GFP (pBMN-I-GFP) or Ds-Red as previously described.

For the Cytodex-3 cell culture, fifty mg of Cytodex-3 microcarrier beads (Sigma, St. Louis, Mo., USA) were hydrated with 5 mL phosphate buffered saline (PBS) —Ca²⁺/—Mg²⁺ (Hyclone) for four hours with occasional mixing to avoid aggregation. PBS solution was removed and washed out with freshly prepared 70% ethanol for total of four washes. The last 70% ethanol wash was carried overnight. The following day, ethanol was removed and 10 mL of HepG2 growth media was added for a total of four washes. The last wash was removed and HepG2 cells were passaged into a resuspension of 1×10⁶ cells/ml. 6×10⁶ cells were added to 4 mL of HepG2 media containing Cytodex-3 beads and gently mixed. The bead-cell mixture was added to a 100 mm petri dish (BD Falcon) and incubated for three days at 37° C. and 5% CO₂ for optimal microcarrier coverage.

To form the three-dimensional (3D) constructs, fresh or cultured SVF (10⁶ cells/mL) were suspended into 3 mg/mL of collagen type I (BD Biosciences, San Jose, Calif., USA) and 0.2 mL of the suspension was seeded into wells of 48-well culture plates. Constructs were implanted subcutaneously on the flanks of Rag1 mice as previously described. To assess the potential of fresh and cultured SVF to participate in the neovascularization process, fresh or cultured SVF from GFP rats (10⁶ cells/mL) were seeded into collagen gels concomitantly with isolated FMFs (20,000/mL). FMF/SVF/collagen suspensions were pipetted into wells of a 48-well culture plate (0.2 mL/well) to form a 3D construct that were either cultured in DMEM+10% FBS or implanted subcutaneously on the flanks of Rag1 mice as previously. Alternatively, SVF were seeded in the presence of HepG2 cells before implantation.

To analyze the implants, microvascular constructs were harvested at either 4 or 6 weeks after implantation and fixed in 4% paraformaldehyde for 20 minutes. Samples were permeabilized with 0.5% Triton X-100 and rinsed with PBS. After blocking for two hours with 10% goat serum (Sigma), samples were incubated overnight with fluorescent or biotin conjugated lectins. Following three 15 minute washes in divalent cation free (DCF)-PBS, samples were imaged en bloc with an Olympus MPE FV1000 Confocal Microscope and analyzed with Amira 5.2 (Visage Imaging, Inc., San Diego, Calif., USA). SVF cells were identified by either constitutive expression of GFP (when obtained from animals that ubiquitously and constitutively express GFP) or labeling with TRITC/Fluorescence conjugated or Cy5-streptavidin GSI (rodent SVF) or UEAI (human SVF) lectin (Vector labs, Burlingame, Calif., USA). To evaluate vessel perfusion in the implanted constructs, host mice were perfused intravenously with the blood tracer dextran-TRITC 2,000,000 MW for 15 minutes before the constructs were harvested. Confocal microscopy images of implants (from 3-12 image stacks per each of 5 implants) with HepG2-GFP⁺ clusters were identified and examined for presence of GS1-Cy5⁺ vasculature, DiI-LDL, or both. Those images without HepG2-GFP⁺ clusters were not included. Significant differences between HepG2-GFP⁺ clusters with both GS1-Cy5⁺ vessels and DiI-LDL and those with either one or the other or none were determined using a two-tailed t-test between the sample pairs of interest. To determine if DiI-LDL uptake is correlated with the presence of GS1-Cy5⁺ vasculature, HepG2-GFP⁺ clusters positive for DiI-LDL were plotted against those clusters positive for GS1-Cy5 vasculature. The Pearson correlation coefficient was then calculated for statistical correlation between the two variables. Significant differences in measured parameters between fresh and cultured SVF cells (n=3/condition) was determined by a Student's t-test with a normality check.

Results

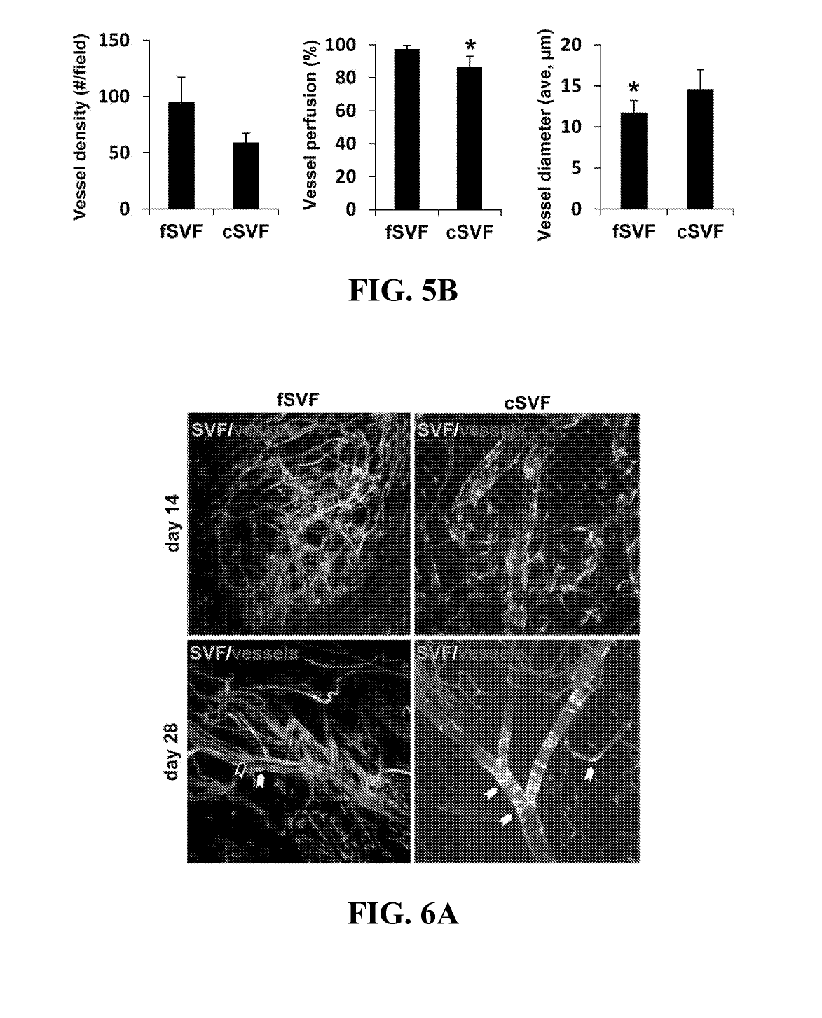

One of the technical hurdles for developing a functional tissue mimic or cell system is providing a vascular interface between the host circulation and implanted parenchymal cells. The freshly isolated stromal vascular fraction (SVF) from adipose is rich in vascular and other relevant cells capable of incorporating into vessels in vivo. Similarly, cultured SVF cell populations also exhibit vascularizing potential, supporting the use of both fresh and cultured SVF cells (fSVF and cSVF, respectively) as cell sources in transplantation therapies. Based on those observations, and without wishing to be bound by any particular theory, it was believed that adipose SVF cells alone are capable of forming de novo a new vasculature that was amenable to use in vascularizing a tissue mimic. To test that belief, SVF cell preparations from transgenic rats ubiquitously expressing GFP were used to form implants, and it was observed that both fSVF and cSVF cells in a 3D collagen matrix free of exogenous growth factors self-assembled to form a perfused vasculature (FIG. 5A). For both SVF cell preparations, complete vascular trees consisting of arterioles, capillaries and venules were observed and comprised entirely of GFP⁺ cells, indicating an SVF origin. While both fSVF and cSVF generated perfused vasculatures, those formed by cSVF had lower vessel densities than fSVF-derived vasculatures (fSVF, 94.9±22; cSVF, 59.2±8 vessels/field of view) and total vessel perfusion was significantly less, (fSVF, 97.4±0.8; cSVF, 86.7±1.9) (FIG. 5B). Additionally, the average vessel diameter within the cSVF-formed vasculatures was significantly higher suggesting a lower proportion of smaller capillary-like diameters than in fSVF-formed vasculatures (fSVF, 11.7±1.5; cSVF, 14.6±2.3) (FIG. 5AB).

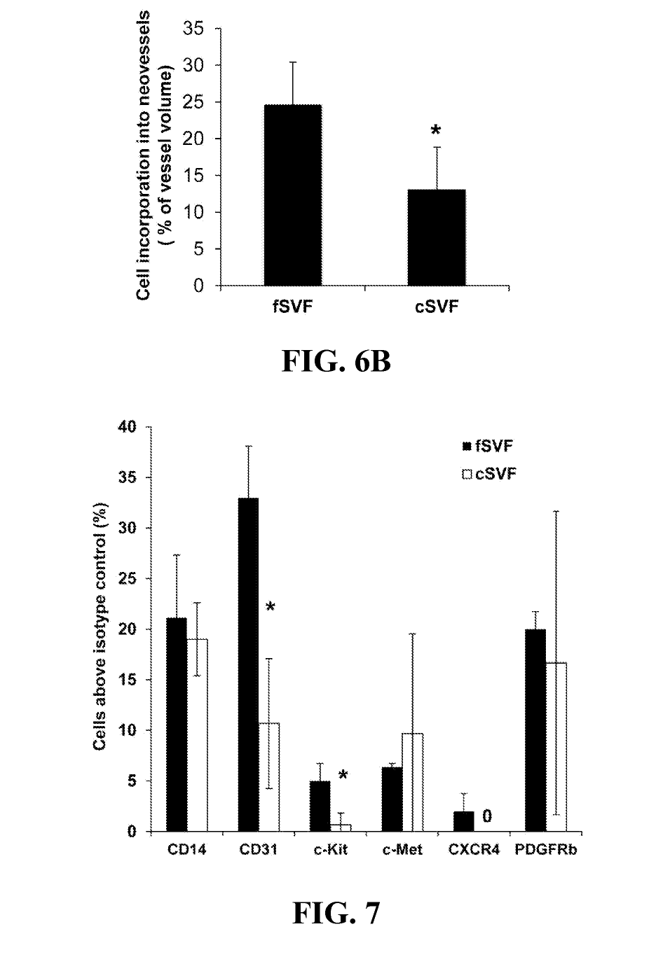

Another issue with functionalizing an implanted tissue mimic is efficient integration between the mimic-host vasculatures. Using an experimental model of neovascularization involving the implantation of angiogenic microvessels, the ability of SVF cells to incorporate into an angiogenic vascular bed, an activity essential to vascular integration, was next investigated . As with de novo vessel assembly, both fresh and cultured SVF cells participated in the formation of new vessel elements during active angiogenesis (FIG. 6A). During the early phases of neovascularization, which is dominated by angiogenesis and immature network formation, SVF cells were intimately associated with the nascent, endothelial cell-derived neovessels throughout the developing neovasculature. In the later implants, the mature vasculatures that formed were comprised of GFP⁺ (i.e., SVF-derived) and GFP-negative (i.e. non-SVF-derived) cells (FIG. 6A). Moreover, many of the non-SVF-derived vessels were populated with SVF cells or were chimeras of non-SVF-derived and SVF-derived vessel segments (FIG. 6A). In mature angiogenic implants containing fSVF, GFP⁺ cells were observed in endothelial and perivascular positions of all vessel types. In contrast, cSVF cells were found predominately in perivascular positions and rarely in the endothelial position. In addition, the extent of cSVF cell incorporation into the formed vasculature was approximately half that of fSVF cells (fSVF, 24.6±10.4%; cSVF, 13±6.6%) (FIG. 6B).

The above-described differences in incorporation potential and vascular position indicated that submitting SVF cells to culture promotes either a selection of a perivascular phenotype or changes in the population potential. To investigate the different cell populations present in fresh and cultured SVF, the expression of different cell type markers was assessed by flow cytometry (FIG. 7). Consistent with the vascularizing potential and predicted from a related study, cSVF cell population contains less than half the number of CD31⁺ cells (presumably endothelial cells) than fSVF cells. Similarly, the proportion of c-Kit⁺ progenitor cells was greatly reduced in cSVF cells as compared to fSVF cells. However, the proportions of cells expressing markers for monocyte/macrophages (CD14), perivascular cells (PDGFR-β) and multipotent cells (CXCR4, c-Met) was not different. The similar presence of PDGFR-β⁺ cells in both SVF preparations might explain the shared potential for establishing mural/perivascular coverage of the new vessel elements.

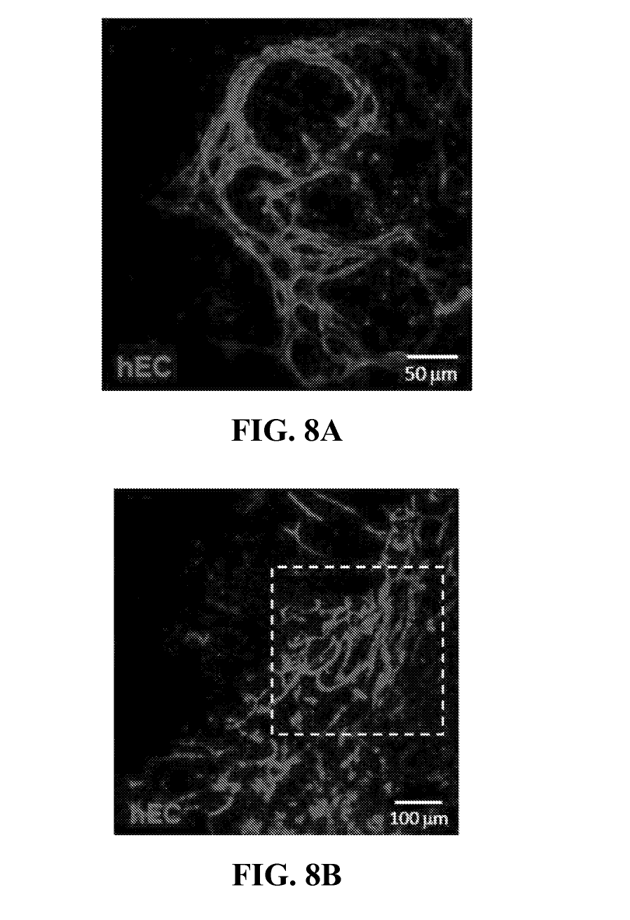

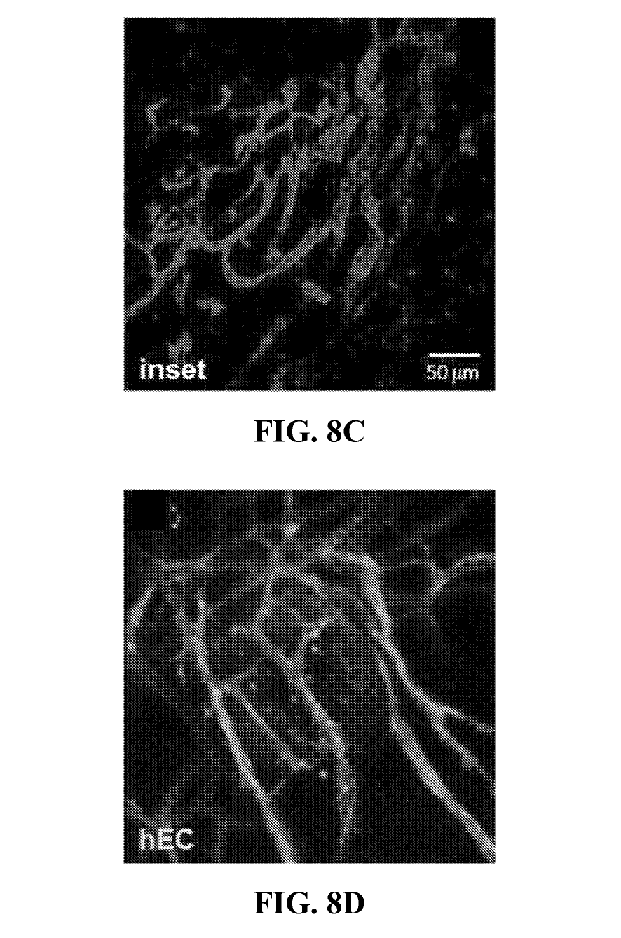

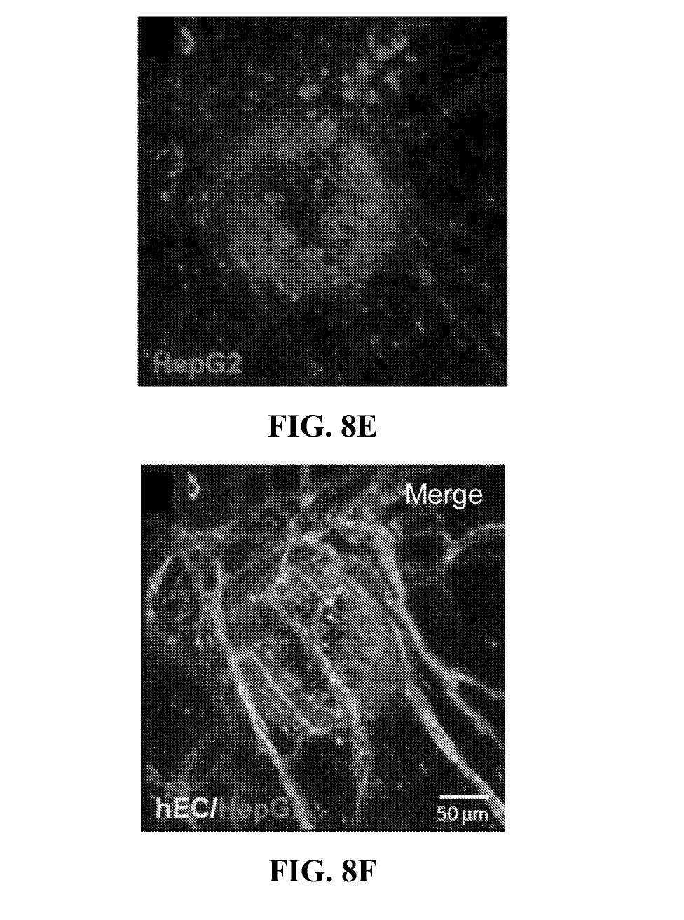

Having demonstrated the vascularizing potential of SVF cells using a transgenic lineage marker, the vascularizing potential of clinically relevant human SVF cells was next determined. In this regard, the above de novo assembly experiments were repeated using freshly isolated SVF cells derived from discarded lipo-aspirates with the exception that the SVF cells in collagen constructs were implanted for 6 weeks instead of 4 weeks. As with the rat SVF cells, the human SVF cells were also able to self-assemble into a vascular network, although the human SVF-derived network may still be undergoing neovascular remodeling at this time (FIGS. 8A-8F). To determine if the human SVF cells retained this ability to assemble a vasculature de novo in the presence of parenchyma cells, constructs containing human SVF with HepG2 cells, a hepatocyte-like cell line, grown on Cytodex-3 beads were implanted to maintain the hepatocyte-like phenotype in the 3D environment. As before, the human SVF cells assembled a vascular network in these implants. Interestingly, human SVF-derived vessel networks formed around and in close approximation to the HepG2 clusters.

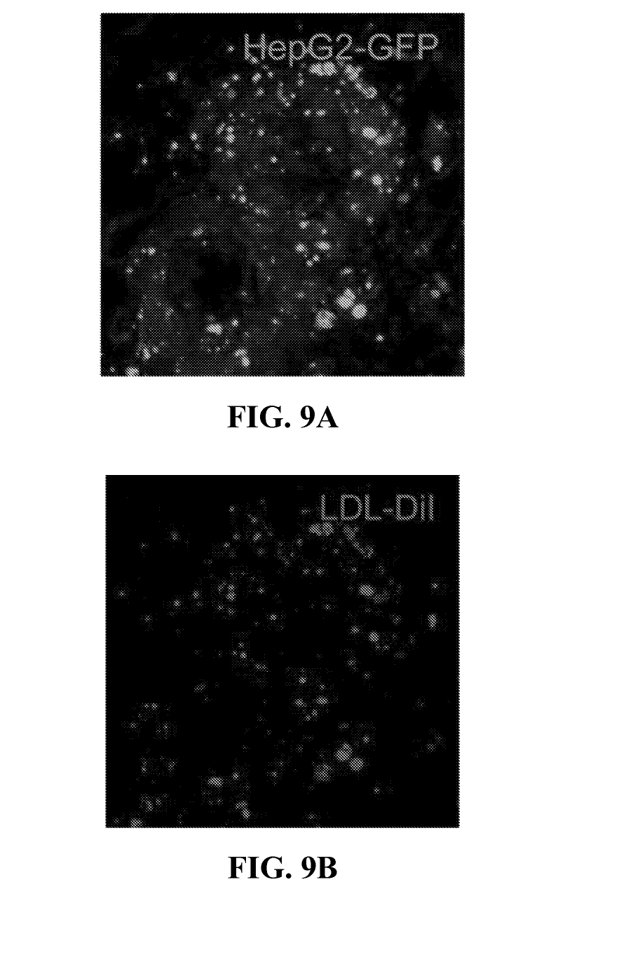

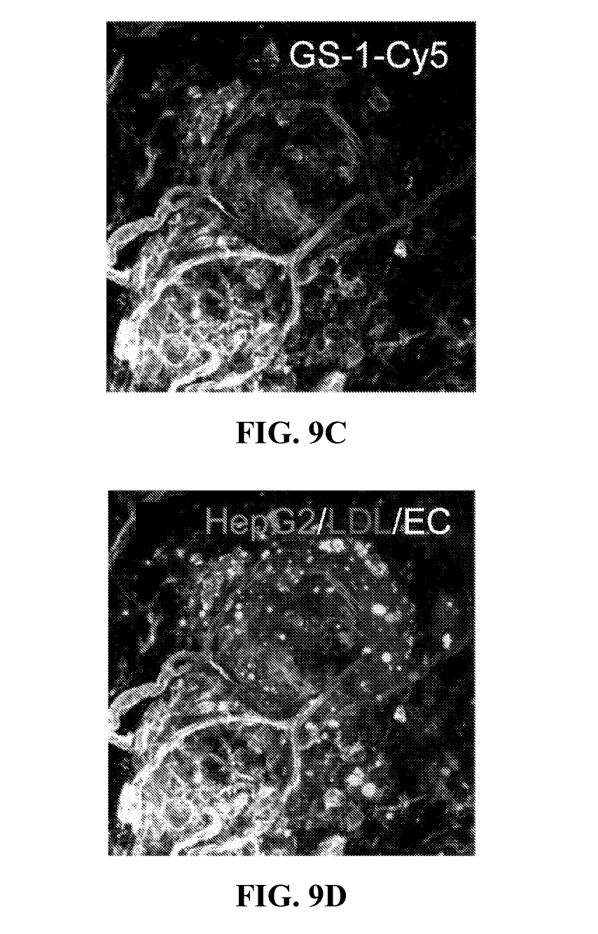

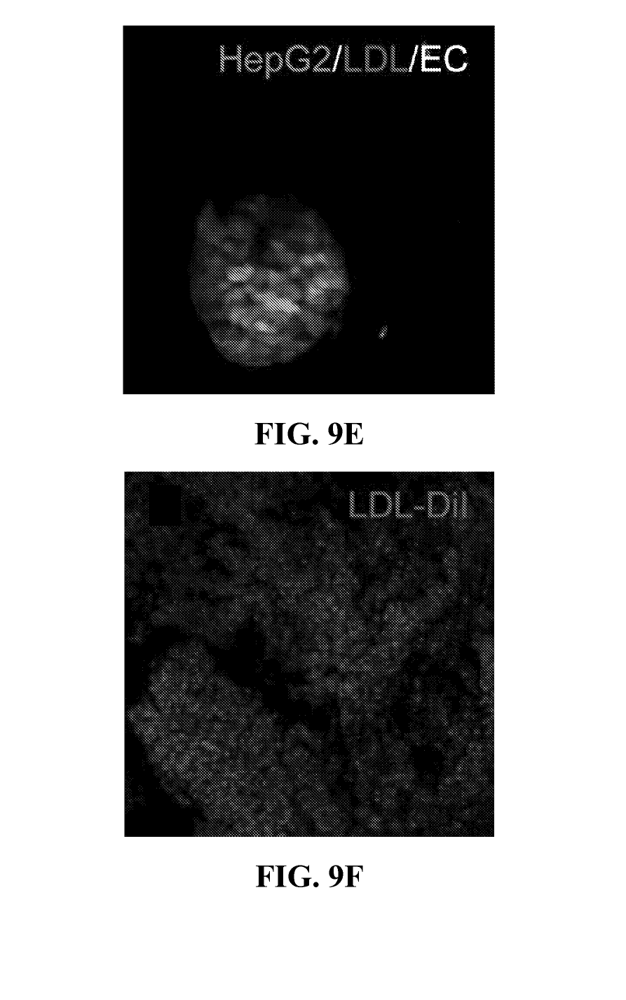

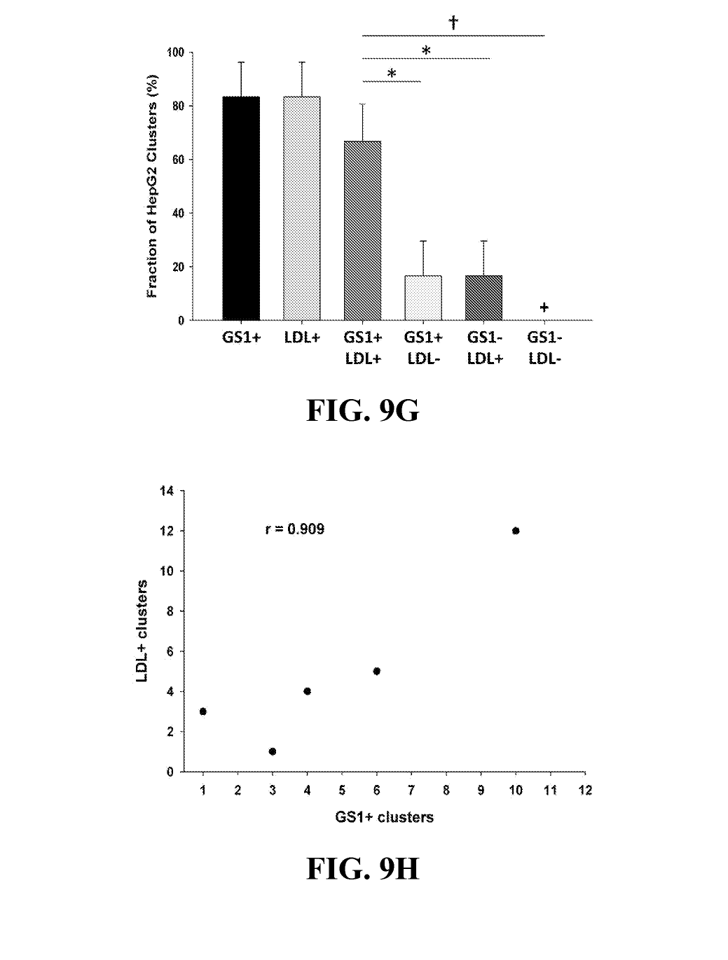

Because of the close association between SVF cell-derived vessels and HepG2 clusters, it was next determined if the vascularized cell system was functional. To do this, the fact that HepG2 cells express the LDL receptor and take up LDL similar to mature hepatocytes was taken advantage of by examining LDL uptake in the vascularized implants. As expected, HepG2 cell implants vascularized with fresh SVF cells took up DiI-labeled LDL (DiI-LDL) injected intravenously into the host mouse (FIGS. 9A-9D). Approximately 83% of the HepG2 clusters were associated with a vascular network or DiI-LDL uptake, while approximately 67% of the HepG2 clusters were associated with both. Further analysis indicated a strong correlation (r=0.909) between the presence of vessels and DiI-LDL uptake by HepG2 cell clusters. Indeed, HepG2 clusters not associated with a vasculature did not co-localize with DiI-LDL despite DiI-LDL uptake by host liver.

With the above-described strategy, a functional, vascularized tissue mimic was generated by combining parenchymal and adipose-derived SVF cells. In the foregoing experiments, the tissue mimic was a model liver module using a human model hepatocyte cell line (HepG2) as the parenchyma. Included this strategy was the ability of adipose-derived SVF cells (either freshly isolated or cultured) to spontaneously form de novo a mature microvasculature. The uptake of LDL by the HepG2 cells also demonstrated that this formed microvasculature served as a functional vascular interface between the host circulation and the parenchymal cells. The vascular-parenchyma integration observed in the SVF-based implant, intrinsic to native tissues, highlighted the therapeutic potential of the implant design/strategy. Further, although the above-described experiments were directed toward a liver tissue mimic, the use of adipose SVF cells was an enabling solution with broad applicability. Related to this and due to the inherent vascularization ability of isolated adipose SVF cells, a point-of-care strategy was thus believed to be possible whereby freshly harvested SVF cells from readily acquired lipoaspirates can be used in an autologous fashion. Additionally, given that cultured SVF cells retain the ability to form de novo blood-perfused vasculatures, a more therapeutically convenient “off-the-shelf” approach could be employed by using banked, pooled adipose SVF cells expanded by culture. The low immunogenicity of adipose-derived cells makes the allogeneic approach feasible. This immune-privileged aspect of adipose SVF cells can even facilitate the use of allogeneic parenchymal cells in the implant design should an autologous solution not be available. Finally, multiple Phase I clinical trials using different adipose-derived SVF preparations as a source for therapeutic mesenchymal cells indicate that these cells are very safe.

Previous attempts towards the development of vascularized liver grafts for transplantation consisted of incorporating vascular endothelial growth factor into scaffolds to enhance vascularization of transplanted hepatocytes In the foregoing studies, however, it was demonstrated that when combined with HepG2 parenchymal cells, SVF cell-derived vasculatures envelop these cells, forming a functional interface. Indeed, the effective integration of transplanted liver tissue mimics was demonstrated six weeks post-implantation through the metabolic interaction between SVF formed vessels and parenchyma cells, as illustrated by the uptake of fluorescently labeled LDL by HepG2 cells. That observation indicates that other therapeutic cells could be combined with SVF to form modular tissue mimics for delivery or removal of circulating biomolecules.

The liver tissue mimic described above was developed as a modular system designed to perform a specific function (LDL uptake in this case). However, tissue mimic modules with different functional purposes can also be assembled by incorporating different parenchymal cells along with the vascularizing adipose SVF cells. In this way, via the modular approach described herein, more complex organoids capable of performing multiple, potentially integrated, physiological functions could be generated by combining these different multiple tissue mimics. The modular strategy was also believed to be scalable by simply implanting more or less of the modules to meet therapeutic need. Additionally, select modules (or all) could be removed should there be an unexpected, deleterious outcome to the implantation (e.g. infection). Depending on the configuration, these mimics, such as the liver mimic described herein, could prove useful not only as an implantable functional replacement (e.g. LDL clearance) for regenerative medicine, but also as a human model tissue system for triaging/developing drug candidates targeting specific parenchyma types, evaluating drug metabolism (as with the hepatocyte-like module), and other translational and mechanistic investigations.

While the inherent vascularization capability of adipose SVF cells is maintained in early passage culture, the capacity of these cells to incorporate into vascular sites of neovascularization (i.e. angiogenic neovessels) was altered, suggesting that culturing has an effect on the SVF cells. This was demonstrated not only by the significant decrease in SVF incorporation into formed neovessels but also by the position of the incorporated cells (endothelial and perivascular for fresh SVF; mostly perivascular for cultured SVF). Flow cytometry of select markers revealed a significant decrease in the percentage of CD31⁺ and cKit⁺ cells after culture, suggesting a reduction in the proportion of endothelial cell phenotypes. This reduction corresponded to a lower density (i.e. number) of vessels formed de novo by the cultured SVF cells and was consistent with the idea that the endothelial cells present in an adipose SVF cell isolate are required for vascular assembly. Interestingly, the proportion of cells with perivascular phenotype (PDGFR-β⁺ cells) did not change with culture. Again, this was consistent with the observation that cultured SVF cells preferentially incorporated into the mural position in angiogenic neovessels.

One aspect to note was that the plating and culture conditions that were employed differed from those used by others selecting for adipose-derived stem cells (ADSC). While there were cells expressing mesenchymal stem cell-like markers in early-passages of cultured SVF cells, mixed cell phenotypes were present that were not typically observed in the other reported ADSC phenotypes. Those mixed phenotypes observed in the cultured SVF cells may explain why the cultured SVF cells were able to generate de novo a vasculature (as all necessary cell types appear to be present), albeit to a lesser extent than with the freshly isolated SVF cells.

Another aspect of the current study was the ability of SVF cells, either fresh or cultured, to go from a single-cell suspension to a self-assembled functionally mature vasculature. Endothelial cells can play a role in this process. However, endothelial cells alone are insufficient to form a mature vasculature either in vitro or in vivo. Non-endothelial support cells are required to achieve vessel stabilization and maturation. Within the SVF are these support cells, such as perivascular cells and/or mesenchymal stem cells. But, also other stromal cells present in the isolate, such as fibroblasts and macrophages, can be important. Although it is possible that vascular beds from all tissues, when isolated and disassembled, would show the same self-assembly capacity as demonstrated here by adipose-derived SVF cells, the adipose vasculature has been proposed to be evolutionarily less mature than other more quiescent vascular beds and thus more plastic. Without wishing to be bound by any particular theory, it was believed that perhaps that plasticity was important to allow tissue, and thus vascular, remodeling in response to the energy storage requirements of adipose tissue. Besides its relative abundance and accessibility compared to other adult cell sources, the above results highlight adipose-derived SVF clinical utility for vascularization under a variety of relevant conditions.

In summary, the foregoing experiments demonstrate that adipose SVF cell-derived vasculatures from rodent and human sources can effectively integrate with host vessels and interface with parenchymal cells to form a functional, implanted tissue mimic with therapeutic potential. This enabling technology can also be expanded to generate a variety of tissue mimics and cellular modules by changing the parenchymal cell type (e.g. cardiomyocytes, β-cells, or engineered therapeutic cells). The LDL uptake observation suggests that the adipose-derived vasculatures in these implant modules can acquire functional specificity, an important aspect for therapeutic efficacy and mimic function. This approach whereby abundant therapeutic cells are utilized without selection or further manipulation, beyond the initial isolation process, creates new avenues towards tissue mimic and therapeutic applications including the ability to incorporate disease- and/or patient-specific dynamics.

### Example 4—Cholesterol Scavenging Cell System

In view of the foregoing experiments, for the development of a cell system capable of scavenging cholesterol, referred to herein as a cholesterol scavenging module (CSM), SVF cells are derived from the LDLR-KO mouse and are first transduced using either an episomal plasmid (pEHZ-LDLR-LDLR) that contains 10 kb of upstream regulatory sequences for physiological control of LDLR expression (see Hibbitt, et al., Long-term Physiologically Regulated Expression of the Low-density Lipoprotein Receptor In Vivo Using Genomic DNA Mini-gene Constructs, Molecular Therapy (2010) 18(2), 317-326, which is incorporated herein by this reference) or a lentivirus construct containing the human LDLR (pLenti-LDLR, FIG. 10). The vector is based on the Gateway® (Invitrogen) technology that allows for rapid sequence insertion. The LDLR sequence is from the ORFeome database and cloned into a Gateway® Entry vector (pEntr221), as the vector allowed for ease of construction and availability of other sequences for future use, and also allowed for a reduction in the immunogenic response of mice to lentivirus while allowing the versatility necessary for transducing dividing and non-dividing cells.

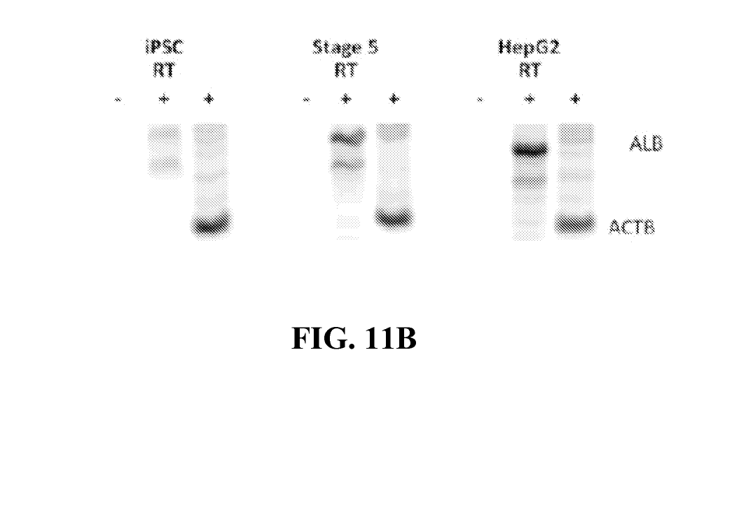

In preparing the CSM, one embodiment includes the generation of hepatocyte-like cells (HLC) from induced pluripotent stem cells (iPSC). In these embodiments, human iPSC are generated from the fetal lung fibroblast cell line IMR90 using lentiviral vectors for POU5F1, NANOG, SOX2, KLF4, LIN28 and MYC. iPSC (FIG. 11A, left) are then transitioned to a feeder free culture and differentiated to HLC using a five stage protocol (FIG. 11A, right). Briefly, iPSC were cultured on Matrigel™ (BD Biosciences) in either 20% KSR-MEF conditioned media or mTeSR1 and grown to confluence. That media was then replaced with definitive endoderm induction media (DEIM, RPMI1640, 0.5 mg/ml albumin Fraction V, 100 ng/ml activin-A) for 24 hours, then 0.1 and 1% insulin-transferrin-selenium (ITS) was supplemented to the DEIM on days 2 and 3, respectively (Stage 1). After 3 days, the media was changed to Hepatocyte Culture Medium (HCM, Lonza) supplemented with 30 ng/ml FGF4 and 20 ng/ml BMP4 for 4 days (Stage 2). Next, the media was changed to HCM with 20 ng/ml HGF and 20 ng/ml KGF for 6 days (Stage 3). The HCM was then supplemented with 10 ng/ml oncostatin-M, 0.1 μM dexamethasone for 5 days (Stage 4). Finally, the media was changed to DMEM with N2, B27, L-glutamine, 1% nonessential amino acids and 0.1 mM 2-mercaptoethanol for 3 days (Stage 5). Subsequent PCR analysis of albumin transcription (ALB) demonstrated the phenotype change from an undifferentiated pluripotent cell not expressing ALB to a HLC positive for ALB transcription (FIG. 11B). HepG2 was used as positive control, no RT was the negative control and R-actin was the control for loading.

Subsequent to the generation of the above data, autologous cell-based implantable apheresis systems are then developed. From the data, a three-dimensional collagen I construct is initially used for the analysis of microvascular assembly and remodeling. Briefly, for the source of SVF cells, female retired breeders are used for adipose isolation. Adipose tissue from the uterine horns is isolated and minced to a paste consistency and is then fully digested with collagenase and centrifuged. The resulting SVF cells are then plated on 1% gelatin coated tissue culture flasks in Rat Complete media (DMEM-HG, 10% FBS, 1 mM Hepes, 80 μg/ml ECGS, L-glutamine and pen/strep). Cells are allowed to adhere for 45 min and the non-adherent cells are then washed away. The remaining adherent cells are then grown to confluence representing P0. Cultured SVF cells are used from P0 to P4 for CSM generation. It has been determined that cultured SVF cells retain the functional capacity to integrate into the generated microvasculature, and thus, no reduction in vascularization is expected by using both fresh and cultured SVF cells.

Three sources are initially tested for the cholesterol scavenging module (CSM), all originating from adipose SVF cells isolated from the LDLR-KO mouse (Table 1). For CSM1, SVF cells transduced with pLenti-LDLR (SVF-LDLR) are tested to determine if those cells can function within the construct to clear LDL-c. CSM2 are then tested to determine if SVF-LDLR can be differentiated to a hepatocyte-like cell (HLC) as has been described previously. As a third test, SVF-LDLR are reprogrammed to iPSC by transduction with lentiviral vectors, then differentiated to HLC as described above. As a negative control, pLenti-Empty transduced SVF cells are used in parallel experiments.

TABLE 1
Sources of Cholesterol Scavenging Modules.

| CSM # | Generation Strategy |
|---|---|
| CSM1± | SVF cells transduced w/LDLR or Empty vector |
| CSM2± | SVF cells transduced w/LDLR or Empty vector → |
| | HLC |
| CSM3± | SVF cells transduced w/LDLR or Empty vector → |
| | iPSC → HLC |

For pLenti-LDLR transduction, the LDLR transduction vector titer is determined using 293FT cells and the titered virus is tested on SVF cells to determine the multiplicity of infection (MOI) required for maximum transduction. That standard is then maintained for all LDLR and empty transductions to maintain a uniform expression of LDLR across CSM platforms and experiments.

For iPSC generation and culture, transduced SVF cells (or Empty) are induced to reprogram the cells to iPSC using a mix of four lentiviral vectors, as previously described, and containing the reprogramming genes POU5F 1, NANOG, SOX2 and MYC. In this regard, SVF cells are cultured in 20% KSR media (DMEM/F12, 20% Knock-Out Serum Replacement (KSR), 1×MEM-NEAA, 1×pen/strep, 10 ng/ml bFGF, 0.1 mM (β-mercaptoethanol). Colonies are tested for expression of alkaline phosphatase, Oct4, Nanog, SSEA1, Tra-1-60 and Tra-1-81. Colonies are formed into embryoid bodies or implanted for teratoma formation and examined by histology for formation of all three germ layers. For expansion culture, iPSC are grown on MEF derived from CF1 mice and inactivated with Mitomycin-C (23) in 20% KSR media. For feeder-free culture, iPSC are passaged onto hESC qualified Matrigel and cultured in MEF conditioned media. Established clonal colonies of iPSC are tested for karyotype and DNA fingerprinted for lineage confirmation to the LDLR-KO mice.

For HLC generation, individual protocols are used for generation of HLC depending upon beginning cell type. For CSM2 and the differentiation of SVF cells to HLC, the protocol published by Banas, et al. is used in a 3-Stage process. For iPSC, the protocol of Song, et al. is used in a 5-Stage protocol that has been used on human iPSC for HLC derivation (see FIG. 11A).

For HLC characterization, and because the HLC is being derived, it is thought to be important to define the general characteristics of the cell. As such, the transcription profile of hepatocyte associated genes is tested by PCR, including the testing of genes such as ALB, AFP, CK8, CK18, HNF4a and HNF6. Protein expression is examined by Western blot, immunocytochemistry and ELISA. Uptake of LDL-c is also tested by quantitative fluorescence assay of Dil-LDL and HLC uptake of Dil-LDL is tested under static and dynamic conditions. For dynamic testing, a microfluidics chamber designed by the Roger Kamm lab of MIT is used, where HLC is cultured in a 3D collagen I gel and subjected to low-interstitial flow levels (˜30 μm/min). The 3D construct can then be imaged by confocal microscopy, the image volume rendered and LDL uptake quantified.

The apheresis cell-based system itself is then fabricated in three configurations (Table 2). For all constructs, collagen I is used at a concentration of 3 mg/ml, with the initial volume of the construct being 200-250 μl to provide a suitable sized construct that can be inserted subcutaneously. For configuration 1 (C1), the different CSM are combined with fresh isolate SVF cells as a single cell suspension to test for random integration and self-assembly in vivo as that test determines if the CSM can self-assemble into a functional unit with the microvasculature formed by SVF cells. Configuration 2 (C2) uses Cytodex beads for pre-attaching CSM and integrating into the construct, which has been shown to enhance survival and functional maintenance of mature hepatocytes. For configuration 3 (C3), the CSM is preformed in vitro with Cytodex beads then combined with fresh SVF cells at implantation.

TABLE 2
Apheresis System Configurations.

| Device | |
|---|---|
| Configuration # | Generation Strategy |
| C1 | CSM + SVF cells, random mix and self assembly |
| C2 | CSM-Cytodex + SVF cells, 3D bead and |
| | self assembly |
| C3 | CSM-Cytodex pre-culture in vitro, |
| | SVF cells mix at implantation |

As an alternative to the foregoing experiments, for the generation of iPSC, a different cell type other than SVF cells can be reprogrammed One potential source is skin fibroblasts that have been isolated from LDLR-KO and are available for use. Another source, is an allogeneic source, such as HepG2 cells.

### Example 5—Efficacy and Safety of Cell-Based Systems for Apheresis

To test the autologous cell-based implantable apheresis systems for efficacy and safety, the efficient clearance of excess LDL-c is assessed, without generating unintended safety issues, along with potential hazards such as teratoma, plaque, and xanthoma formation. First, LDL-c clearance and quantification is assessed for each configuration generated above in triplicate with each animal receiving a construct on the left and right side. To test the function of the cell system, LDL-c serum levels are assessed where at the beginning of each experiment 250 μl of blood/animal is collected. Prior experience with microvascular assembly from SVF cells indicates an immature vasculature is formed by 2 weeks and remodeling and maturation occurs at 4 to 6 weeks and, therefore, blood samples are subsequently collected each 2 weeks up to 6 weeks. For quantification of serum LDL levels, the MaxDiscovery LDL Cholesterol assay is used, which is an enzymatic assay that can be read in a 96-well plate format. All samples are run in triplicate.

To test for localization of LDL-c in the construct and potential migration of construct cells, confocal microscopy is utilized. In this regard, at the end of each experiment, the animal is perfused with GS1-biotinfor endothelium detection and LDL-Dil. The construct and other organs of interest are then processed for confocal or histological analysis. To detect GS1-biotin, samples are incubated with Cy5-streptavidin. Constructs are examined for GFP expression indicated in LDLR transduced cells and co-localization of LDL-Dil. The GFP⁺ CSM and Cy5⁺ staining is used for quantifying vascular structure within the construct and CSM/vascular integration. Constructs are imaged by confocal microscopy, stacks volume rendered and images analyzed using Amira software. In the image analysis of the constructs, vessel structure and maturation state (i.e., vessel density, size, segment length) are examined along with how the CSM and vessels interact, whether the vessels form fenestrae and whether the fenestrae directly associated with CSM, whether the vessel/CSM form sinusoids and whether the LDL-Dil localized to GFP+ cells or elsewhere. Constructs can also be immunostained for the LDLR or other markers of interest.

Configurations of the cell system are then assessed for survival of the system. As an implantable therapeutic in humans and other subjects, it would be optimal for the system to effectively function for as long as possible without replacement. To test survival and function, blood samples are collected on a biweekly schedule to assay LDL-c. The constructs are processed as described at 6 and 12 w, but are also examined for signs of apoptosis using a TUNEL-DAB colorimetric assay. GFP expression, LDL-Dil aggregation and DAB detection are also correlated to the corresponding LDL-c level. Each time point is run in triplicate and repeated three times.

For the implantable apheresis cell system technology to be clinically relevant, it is thought to also advisable to make the system scalable so the construct cell concentration is varied and correlated to obtain LDL-c levels that are appropriate for a given animal weight. To estimate the cell system scalability, three different concentrations, 0.5, 1 and 2×10⁶ total cells/ml are used. After implantation, blood is collected for LDL-c content and constructs harvested for analysis and correlation to cholesterol levels as described. Cell system cell migration is also assessed using harvest animal liver, lung and heart for histological examination for GFP+ cells and LDLR expression.

For the HLC derived from iPSC, the differentiation from iPSC to HLC is also tested to determine whether the system is effective for eliminating teratogenicity. iPSC derived HLC are suspended in Matrigel and injected into the hind leg of Rag1 immune compromised mice in parallel with undifferentiated iPSC. As a secondary test for teratogenicity, iPSC are combined with SVF cells at a 1:1 ratio and implanted subcutaneously in parallel with the apheresis cell systems Animals with iPSC implanted may not be allowed to progress the entire experiment duration to minimize suffering if tumors form.

Formation LDLR-KO mice do not normally form vascular plaques or xanthomas unless fed high fat diets. However, because LDLR-KO mice exhibit high LDL-c even on normal chow, it is possible the apheresis cell systems of the presently-disclosed subject matter could become a lipid plaque or lead to xanthoma since the systems are implanted subcutaneously. To assess the possible phenomenon, animals receive either the optimized apheresis system or an implant with SVF cells only and fed a normal chow diet for 6w. Constructs are then harvested and processed for histology and immunostained for GS1, LDLR, CD68 (macrophage marker) and Nile Red (lipid detection).

Confocal images are also generated using an Olympus BX61SWI laser scanning confocal microscope. Image stacks are imported into NIH ImageJ, converted to 8-bit grayscale, stack attributes noted, and saved for further processing in a commercial image processing software—Amira (Visage Imaging) as originally described by Krishnan et al. Images are corrected for imaging depth, deconvolved, median filtered, and binarized using an automatically generated threshold value for each image stack in Matlab (MathWorks Inc). These binarized images are segmented and size filtered to remove very small debris and skeletonized. Skeletonized data is parsed by a custom C++ program—WinFiber3D (Musculoskeletal Research Labs, University of Utah, Salt Lake City, Utah) as described previously. The 3D coordinates from the skeletonized data are evaluated to obtain the total number of vessels, the number of branch points, the total number of end points, segment (section between two nodes—branch or end), vessel lengths and diameters. Images are acquired using sequential scanning and co-contact points determined from skeletonized images as described. For quantitative image analysis, the use of different imaging depths may necessitate a comparison of image stack volumes to rule out and accommodate for imaging volume bias. In this regard, the total stack volume is first internally normalized by setting the lowest volume to 1, and this number is used to normalize data from the corresponding image stack as appropriate. Normalized data is compared in SigmaStat (Systat), using student t-tests and 2-way ANOVAs or its non-parametric equivalent, the Mann-Whitney U test, where the assumptions of normality and equal variance are violated.

Based on the foregoing experiments, it is observed that the cell systems of the presently-disclosed subject matter are capable of LDL-c uptake and metabolism with little or no residual lipid accumulation. Additionally, it is observed that due to the modularity of the systems, the lipid scavenging capacity can be modulated by the introduction of more or fewer systems. The accumulation of lipid and xanthoma formation can then be used as a visual indicator of need for extracting and replacing the module.

Throughout this document, various references are mentioned. All such references are incorporated herein by reference, including the references set forth in the following list:

### REFERENCES

1. Fox, I. J. & Roy-Chowdhury, J. Hepatocyte transplantation. J. Hepatol. 40, 878-886 (2004).

2. Strom, S. C., et al. Transplantation of human hepatocytes. Transplant. Proc. 29, 2103-2106 (1997).

3. Habibullah, C. M., Syed, I. H., Qamar, A. & Taher-Uz, Z. Human fetal hepatocyte transplantation in patients with fulminant hepatic failure. Transplantation 58, 951-952 (1994).

4. Nagata, H., et al. Prolonged survival of porcine hepatocytes in cynomolgus monkeys. Gastroenterology 132, 321-329 (2007).

5. Kaully, T., Kaufman-Francis, K., Lesman, A. & Levenberg, S. Vascularization—the conduit to viable engineered tissues. Tissue Eng. Part B Rev. 15, 159-169 (2009).

6. Chiu, L. L., Iyer, R. K., Reis, L. A., Nunes, S. S. & Radisic, M. Cardiac tissue engineering: current state and perspectives. Front. Biosci. 17, 1533-1550 (2012).

7. Jain, R. K. Molecular regulation of vessel maturation. Nat. Med. 9, 685-693 (2003).

8. Markkanen, J. E., Rissanen, T. T., Kivela, A. & Yla-Herttuala, S. Growth factor-induced therapeutic angiogenesis and arteriogenesis in the heart—gene therapy. Cardiovasc. Res. 65, 656-664 (2005).

9. Gimble, J. M., Katz, A. J. & Bunnell, B. A. Adipose-derived stem cells for regenerative medicine. Circ. Res. 100, 1249-1260 (2007).

10. Gimble, J. M., Bunnell, B. A., Chiu, E. S. & Guilak, F. Concise review: Adipose-derived stromal vascular fraction cells and stem cells: let's not get lost in translation. Stem Cells 29, 749-754 (2011).

11. Han, J., et al. Adipose tissue is an extramedullary reservoir for functional hematopoietic stem and progenitor cells. Blood 115, 957-964 (2010).

12. Yoshimura, K., et al. Characterization of freshly isolated and cultured cells derived from the fatty and fluid portions of liposuction aspirates. J. Cell. Physiol. 208, 64-76 (2006).

13. Rehman, J., et al. Secretion of angiogenic and antiapoptotic factors by human adipose stromal cells. Circulation 109, 1292-1298 (2004).

14. Koh, Y. J., et al. Stromal vascular fraction from adipose tissue forms profound vascular network through the dynamic reassembly of blood endothelial cells. Arterioscler. Thromb. Vasc. Biol. 31, 1141-1150 (2011).

15. Cervelli, V., et al. Application of enhanced stromal vascular fraction and fat grafting mixed with PRP in post-traumatic lower extremity ulcers. Stem Cell Res. 6, 103-111 (2011).

16. Premaratne, G. U., et al. Stromal vascular fraction transplantation as an alternative therapy for ischemic heart failure: anti-inflammatory role. J. Cardiothorac. Surg. 6, 43 (2011).

17. Leblanc, A. J., Touroo, J. S., Hoying, J. B. & Williams, S. K. Adipose stromal vascular fraction cell construct sustains coronary microvascular function after acute myocardial infarction. Am. J. Physiol. Heart Circ. Physiol. 302, H973-982 (2012).

18. Williams, S. K., Wang, T. F., Castrillo, R. & Jarrell, B. E. Liposuction-derived human fat used for vascular graft sodding contains endothelial cells and not mesothelial cells as the major cell type. J. Vasc. Surg. 19, 916-923 (1994).

19. Cardinal, K. O., Bonnema, G. T., Hofer, H., Barton, J. K. & Williams, S. K. Tissue-engineered vascular grafts as in vitro blood vessel mimics for the evaluation of endothelialization of intravascular devices. Tissue Eng. 12, 3431-3438 (2006).

20. Traktuev, D. O., et al. A population of multipotent CD34-positive adipose stromal cells share pericyte and mesenchymal surface markers, reside in a periendothelial location, and stabilize endothelial networks. Circ. Res. 102, 77-85 (2008).

21. Lois, C., Hong, E. J., Pease, S., Brown, E. J. & Baltimore, D. Germline transmission and tissue-specific expression of transgenes delivered by lentiviral vectors. Science 295, 868-872 (2002).

22. Nunes, S. S., et al. Angiogenic potential of microvessel fragments is independent of the tissue of origin and can be influenced by the cellular composition of the implants. Microcirculation 17, 557-567 (2010).

23. Nunes, S. S., Rekapally, H., Chang, C. C. & Hoying, J. B. Vessel arterial-venous plasticity in adult neovascularization. PLoS One 6, e27332 (2011).

24. Nunes, S. S., et al. Implanted microvessels progress through distinct neovascularization phenotypes. Microvasc. Res. 79, 10-20 (2009).

25. Knowles, B. B., Howe, C. C. & Aden, D. P. Human hepatocellular carcinoma cell lines secrete the major plasma proteins and hepatitis B surface antigen. Science 209, 497-499 (1980).

26. Agius, L., Battersby, C. & Alberti, K. G. Monolayer culture of parenchymal rat hepatocytes on collagen-coated microcarriers. A hepatocyte system for short- and long-term metabolic studies. In Vitro Cell. Dev. Biol. 21, 254-259 (1985).

27. Pak, Y. K., et al. Activation of LDL receptor gene expression in HepG2 cells by hepatocyte growth factor. J. Lipid Res. 37, 985-998 (1996).

28. Pinheiro, C. H., et al. Local injections of adipose-derived mesenchymal stem cells modulate inflammation and increase angiogenesis ameliorating the dystrophic phenotype in dystrophin-deficient skeletal muscle. Stem Cell Rev. 8, 363-374 (2012).

29. Takahashi, K., et al. Induction of pluripotent stem cells from adult human fibroblasts by defined factors. Cell 131, 861-872 (2007).

30. Cai, J., et al. Directed differentiation of human embryonic stem cells into functional hepatic cells. Hepatology 45, 1229-1239 (2007).

31. Mizuno, H., Tobita, M. & Uysal, A. C. Concise review: Adipose-derived stem cells as a novel tool for future regenerative medicine. Stem Cells 30, 804-810 (2012).

32. Kedem, A., et al. Vascular endothelial growth factor-releasing scaffolds enhance vascularization and engraftment of hepatocytes transplanted on liver lobes. Tissue Eng. 11, 715-722 (2005).

33. Broudy, V. C., et al. Human umbilical vein endothelial cells display high-affinity c-kit receptors and produce a soluble form of the c-kit receptor. Blood 83, 2145-2152 (1994).

34. Lammie, A., et al. Expression of c-kit and kit ligand proteins in normal human tissues. J. Histochem. Cytochem. 42, 1417-1425 (1994).

35. Majumder, S., Brown, K., Qiu, F. H. & Besmer, P. c-kit protein, a transmembrane kinase: identification in tissues and characterization. Mol. Cell. Biol. 8, 4896-4903 (1988).

36. Abramsson, A., Lindblom, P. & Betsholtz, C. Endothelial and nonendothelial sources of PDGF-B regulate pericyte recruitment and influence vascular pattern formation in tumors. J. Clin. Invest. 112, 1142-1151 (2003).

37. Bjarnegard, M., et al. Endothelium-specific ablation of PDGFB leads to pericyte loss and glomerular, cardiac and placental abnormalities. Development 131, 1847-1857 (2004).

38. Zhao, Y., Waldman, S. D. & Flynn, L. E. Multilineage co-culture of adipose-derived stem cells for tissue engineering. J. Tissue Eng. Regen. Med. (2012).

39. Boyd, N. L., et al. Dissecting the role of human embryonic stem cell-derived mesenchymal cells in human umbilical vein endothelial cell network stabilization in three-dimensional environments. Tissue Eng. Part A 19, 211-223 (2013).

40. Koike, N., et al. Tissue engineering: creation of long-lasting blood vessels. Nature 428, 138-139 (2004).

41. Stratman, A. N., Malotte, K. M., Mahan, R. D., Davis, M. J. & Davis, G. E. Pericyte recruitment during vasculogenic tube assembly stimulates endothelial basement membrane matrix formation. Blood 114, 5091-5101 (2009).

42. Eto, H., et al. Characterization of Human Adipose Tissue-Resident Hematopoietic Cell Populations Reveals a Novel Macrophage Subpopulation with CD34 Expression and Mesenchymal Multipotency. Stem Cells Dev. 22, 985-997 (2013).

43. Rupnick, M. A., et al. Adipose tissue mass can be regulated through the vasculature. Proc. Natl. Acad. Sci. U.S.A. 99, 10730-10735 (2002).

44. Chang, C. C., et al. Angiogenesis in a microvascular construct for transplantation depends on the method of chamber circulation. Tissue Eng. Part A 16, 795-805 (2010).

45. Hoying, J. B., Boswell, C. A. & Williams, S. K. Angiogenic potential of microvessel fragments established in three-dimensional collagen gels. In Vitro Cell. Dev. Biol. Anim. 32, 409-419 (1996).

46. Boyd, N. L., et al. Microvascular mural cell functionality of human embryonic stem cell-derived mesenchymal cells. Tissue Eng. Part A 17, 1537-1548 (2011).

47. Carmona, R., et al. Immunolocalization of the transcription factor Slug in the developing avian heart. Anat. Embryol. (Berl) 201, 103-109 (2000).

48. Gao, Y., Hu, H. Z., Chen, K. & Yang, J. Z. Primary porcine hepatocytes with portal vein serum cultured on microcarriers or in spheroidal aggregates. World Journal of Gastroenterology 6, 365-370 (2000).

49. Okura, et al, Transplantation of human adipose tissue-derived multilineage progenitor cells reduces serum cholesterol in hyperlipidemic Watanabe rabbits, Tissue Engineering Part C, 17 (2):145, 2011.

50. Grossman, et al, Transplantation of genetically modified autologous hepatocytes into nonhuman primates: feasibility and short-term toxicity, Human Gene Therapy, 3:501, 1992.

51. Grossman, et al, A pilot study of ex vivo gene therapy for homozygous familial hypercholesterolaemia, Nature Medicine, 1(11):1148, 1995.

52. Wade-Martins, et al, Infectious delivery of a 135-kb LDLR genomic locus leads to regulated complementation of low-density lipoprotein receptor deficiency in human cells, Molecular Therapy, 7(5):604, 2003.

53. Kakaei, et al, Liver transplantation for homozygous familial hypercholesterolemia: two case reports, Transplantation Proceedings, 41:2939, 2009.

54. Kroon, et al, The rebound of lipoproteins after LDL-apheresis. Kinetics and estimation of mean lipoprotein levels, Atherosclerosis 152:519, 2000.

55. Song, et al. “Efficient Generation of Hepatocyte-Like Cells from Human Induced Pluripotent Stem Cells,” Cell Research 19: 1233-1242, 2009.

56. U.S. Pat. No. 6,410,314

57. U.S. Pat. No. 6,555,654

58. U.S. Pat. No. 6,610,288

59. U.S. Pat. No. 6,818,439

60. U.S. Pat. No. 6,936,243

61. U.S. Pat. No. 6,962,688

62. U.S. Pat. No. 7,306,794

63. U.S. Pat. No. 7,309,606

64. U.S. Pat. No. 7,323,337

65. U.S. Pat. No. 7,759,118

66. U.S. Pat. No. 7,794,706

67. U.S. Pat. No. 7,932,268

68. U.S. Pat. No. 8,227,249

69. U.S. Pat. No. 8,236,527

It will be understood that various details of the presently-disclosed subject matter can be changed without departing from the scope of the subject matter disclosed herein. Furthermore, the foregoing description is for the purpose of illustration only, and not for the purpose of limitation.

What is claimed is:

1. A cell system for delivering disease-specific therapies, comprising:
a therapeutic cell; and
a plurality of stromal vascular fraction cells.

2. The cell system of claim 1, wherein the therapeutic cell is a parenchymal cell.

3. The cell system of claim 2, wherein the parenchymal cell is a hepatocyte, a cardiomyocyte, or a pancreatic β-cell.

4. The cell system of claim 1, wherein the therapeutic cell is an engineered therapeutic cell.

5. The cell system of claim 1, wherein the engineered therapeutic cell includes one or more genetic modifications for providing missing or deficient gene products.

6. The cell system of claim 5, wherein the engineered therapeutic cell is genetically-modified to express a low-density lipo-protein receptor (LDLR).

7. The cell system of claim 5, wherein the engineered therapeutic cell is genetically-modified to express clotting factor VIII.

8. The cell system of claim 5, wherein the engineered therapeutic cell is genetically-modified to express α1-antiptrypsin.

9. The cell system of claim 1, wherein the engineered therapeutic cell is derived from a stem cell.

10. The cell system of claim 9, wherein the stem cell is an induced pluripotent stem cell.

11. The cell system of claim 1, wherein the therapeutic cell and the plurality of stromal vascular fraction cells are incorporated into a biocompatible matrix.

12. The cell system of claim 11, wherein the stromal vascular fraction cells are present in the biocompatible matrix at a concentration of about 0.5×10⁶ to about 3.0×10⁶ cells/ml.

13. The cell system of claim 11, wherein the biocompatible matrix is comprised of collagen.

14. The cell system of claim 1, wherein the cell system further comprises a microvessel fragment.

15. The cell system of claim 1, wherein the microvessel fragment is isolated from adipose tissue.

16. A cell system for delivering disease-specific therapies, comprising:
a therapeutic cell; and
a stromal vascular fraction cell-derived vasculature.

17. The cell system of claim 16, wherein the therapeutic cell and the stromal vascular fraction cell-derived vasculature are incorporated into a biocompatible matrix.

18. A method of treating a disease characterized by missing or deficient gene products, comprising administering to a subject in need thereof an effective amount of a cell system comprising a therapeutic cell for supplying the missing or deficient gene products and a plurality of stromal vascular fraction cells.

19. The method of claim 18, wherein the disease is familial hypercholesterolemia, and wherein the therapeutic cell expresses a low-density lipo-protein receptor (LDLR).

20. The method of claim 19, wherein the therapeutic cell is genetically-modified to express the low-density lipo-protein receptor (LDLR).

21. The method of claim 18, wherein the disease is hemophilia A, and wherein the therapeutic cell expresses clotting factor VIII.

22. The method of claim 21, wherein the therapeutic cell is genetically-modified to express clotting factor VIII.

23. The method of claim 18, wherein administering the cell system comprises subcutaneously administering the cell system.

24. The method of claim 23, wherein subcutaneously administering the cell system comprises subcutaneously administering the cell system at multiple sites in the body of a subject.

25. The method of claim 18, wherein the therapeutic cell and the plurality of stromal vascular fraction cells are incorporated into a biocompatible matrix.

26. A kit, comprising a therapeutic cell and a plurality of stromal vascular fraction cells.

27. The kit of claim 26, wherein the kit comprises a first vessel including the therapeutic cells and a second vessel including the plurality of stromal vascular fraction cells.

28. The kit of claim 26, wherein the therapeutic cell and the plurality of stromal vascular fraction cells are incorporated into a biocompatible matrix.
